(19) United States
(12) Patent Application Publication
(10) Pub. No.: US 2015/0031898 A1
(43) Pub. Date: Jan. 29, 2015

(54) PROCESS FOR PREPARATION OF PROSTAGLANDIN F2 ALPHA ANALOGUES

(71) Applicant: INSTYTUT FARMACEUTYCZNY, Warszawa (PL)

(72) Inventors: Iwona Dams, Radom (PL); Andrzej Kutner, Warszawa (PL); Michal Chodynski, Pruszkow (PL); Malgorzata Krupa, Warszawa (PL); Anita Pietraszek, Warszawa (PL); Marta Zezula, Warszawa (PL); Piotr Cmoch, Warszawa (PL); Monika Kosinska, Biala Podlaska (PL)

(73) Assignee: INSTYTUT FARMACEUTYCZNY, Warszawa (PL)

(21) Appl. No.: 14/383,543

(22) PCT Filed: Mar. 8, 2013
(86) PCT No.: PCT/PL2013/000028

§ 371 Date: Sep. 6, 2014

(30) Foreign Application Priority Data
Mar. 9, 2012 (PL) P-398388
Mar. 9, 2012 (PL) P-398389

(51) Int. Cl. C07C 405/00; C07F 7/08

(52) U.S. Cl. CPC C07C 405/0016; C07C 405/0041; C07F 7/083; C07F 7/0818
USPC 549/214; 560/121; 564/189; 556/436

(57) ABSTRACT

A convergent synthesis of the prostaglandin F_{2α} analogues, travoprost and bimatoprost, was developed employing Julia-Lythgoe olefination of the structurally advanced phenylsulfone with an enantiomerically pure aldehyde ω-chain synthon. The novel convergent strategy allows the synthesis of a whole series of prostaglandin analogues of high purity from a common and structurally advanced prostaglandin intermediate.

### FIELD OF THE INVENTION

The invention relates to the process for preparation of prostaglandin F_{2α} analogues bearing 13,14-en-15-ol ω-chain possessing 15R or 15S optical configuration at the stereogenic center.

The invention is based on the strategy of a convergent synthesis from a structurally advanced prostaglandin synthon, which enables preparation of a number of synthetic prostaglandin F_{2α} analogues, such as fluprostenol, bimatoprost and travoprost, as well as their epimers and derivatives which may serve as the impurities standards. The prostaglandin F_{2α} analogues are widely used in medical practice, in eye hypertension and open angle glaucoma treatment.

### BACKGROUND OF THE INVENTION

Glaucoma is the eye disease characterized by progressive optical neuropathy and distinctive alteration of optic disc and retina morphology, which cause the decrease of ganglion cells number and diminished field of vision. In the end, these pathological changes result in worsening and irreversible loss of vision. Two types of glaucoma are distinguished, an open angle glaucoma (primary or chronic) and closed angle glaucoma (innate or secondary type). Although the etiology of glaucoma is complex and multifactorial, the increase of intraocular pressure (IOP) damaging the visual nerve has been proved to be the main cause of the illness. Therefore, the standard pharmacological treatment of glaucoma is based on reduction of IOP. Among currently available medications reducing intraocular pressure, β-blockers are usually prescribed at the initial treatment. When there are contraindication to withhold β-blockers, then α-2-simpaticomimetics, the inhibitors of carbonic anhydrase, and next, the latest and the most effective hypotensive medications of the first line treatment, such as prostaglandin analogues and prostamides, are used (N. Ishida and al., Cardiovas. Drug Rev. 2006, 24, 1; G. W. Bean, C. B. Camras, Surv. Ophthalmol. 2008, 53 (Suppl. 1), S69; C. B. Toris and al., Surv. Ophthalmol. 2008, 53 (Suppl. 1), S107).

Endogenous F_{2α} prostaglandin that is not used in medical treatment due to its low specificity, and its synthetic pro-drugs, such as PGF_{2α} isopropyl ester, reduce the intraocular pressure in animals and humans, but also cause conjunctival hyperemia and foreign-body sensation (L. Z. Bito et al., Invest. Ophthalmol. Vis. Sci. 1983, 24, 312; G. Giufreé, Graefe's Arch. Clin. Exp. Ophthalmol. 1985, 222, 139; C. B. Camras et al., Invest. Ophthalmol. Vis. Sci. 1977, 16, 1125; L. Z. Bito, Surv. Ophthalmol. 1997, 41(Suppl. 2), S1). Researchers efforts towards improvement of therapeutic index of the naturally occuring PGF_{2α}, resulted in development of several synthetic PGF_{2α} analogues, such as isopropyl ester of unoprostone, latanoprost, travoprost, bimatoprost and tafluprost, with excellent efficacy and diminished side effects.

The individual response to hypotensive pharmacotherapy in glaucomatous patients is different and due to that there is a continuous demand for a whole range of active PGF_{2α} analogues. Regardless plentiful scientific publications as well as patent applications, that have been published for last decades, the synthetic protocol according to which PGF_{2α} analogues could be obtained effectively, using one structurally advanced prostaglandin intermediate, has not been developed yet. A growing demand for hypotensive PGF_{2α} analogues stimulates the need for the development of a convergent strategy of prostaglandin synthesis that would overcome the disadvantages of a well-known classical Corey synthesis.

Prostaglandin F_{2α} analogues are the structural derivatives of (Z)-7-[(1R,2R,3R,5S)-3,5-dihydroxy-2-[(E,2S)-3-hydroxyoct-1-enyl]cyclopentyl]hept-5-enoic acid, containing two hydroxyl groups at cis position in relation to cyclopentyl ring and two, α and ω, side chains of the relative configuration trans. Different substituents and saturated or unsaturated bonds can be introduced into the α- and ω-side chains. Among the compounds of great importance, prostanoid fluprostenol, its isopropyl ester travoprost, and prostamid bimatroprost, characterized by the presence of 13,14-en-15-ol structure and the presence of the stereogenic center at C-15 of ω-chain, should be mentioned. These prostaglandin analogues are represented by the formulas depicted below:

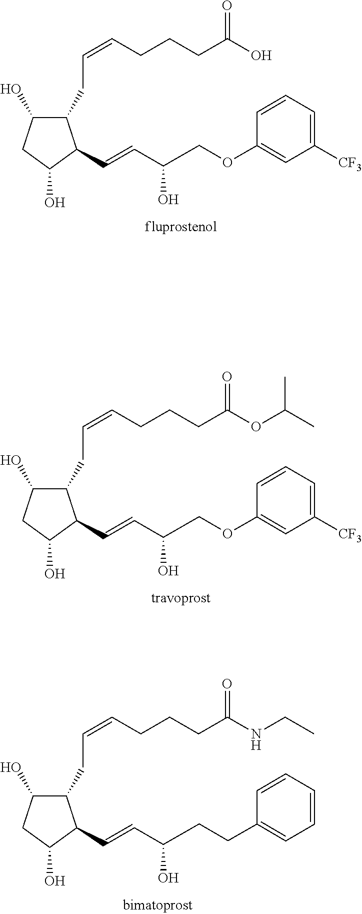 [SMILES: O=C(O)CCC/C=C\C[C@@H]1[C@@H](/C=C/[C@@H](O)COc2cccc(C(F)(F)F)c2)[C@H](O)C[C@@H]1O ; CC(C)OC(=O)CCC/C=C\C[C@@H]1[C@@H](/C=C/[C@@H](O)COc2cccc(C(F)(F)F)c2)[C@H](O)C[C@@H]1O ; CCNC(=O)CCC/C=C\C[C@@H]1[C@@H](/C=C/[C@@H](O)CCc2ccccc2)[C@H](O)C[C@@H]1O]

The above and some other prostaglandin F2α analogues as well as their use for intraocular hypertension and glaucoma treatment have been disclosed, inter alia, in the European Patent Applications EP-A-0170258, EP-A-0253094, EP-A-0364417, EP-A- and EP-A-0639563. The eye drugs approved in glaucoma treatment, including prostaglandin F_{2α} analogues, has been reviewed in M. F. Sugrue, J. Med. Chem. 40 (1997), 2793-2809.

Most synthetic procedures used to attain prostaglandin analogues employ one of the known variants of the Corey method, in which lower and upper side chains are sequentially attached to a derivative of the commercially available (−)-Corey aldehyde/lactone (E. J. Corey et al., J. Am. Chem. Soc. 1969, 91, 5675; E. J. Corey et al., J. Am. Chem. Soc. 1971, 93, 1490; E. J. Corey et al., J. Am. Chem. Soc. 1970, 92, 397; E. J. Corey, Angew. Chem. Int. Ed Engl. 1991, 30, 455). The Corey strategy involves first the attachment of the lower side chain (ω-chain) via a Horner-Wadsworth-Emmons condensation of the Corey-aldehyde ((2S,3R,4S,5R)-4,5-dihydroxy-hexahydrocyclopenta[b]furan-2′-one) with a suitable ketophosphonate (B. M. Trost, Science 1991, 254, 1471). The reaction is generally affected by some drawbacks, such as easy epimerization of labile stereogenic centers (S-W. Hwang at al., Tetrahedron Lett. 1996, 37, 779), and, depending on the base used for deprotonation, formation of additional byproducts (S. Kim at al., Bioorg. Med Chem. Lett. 2005, 15, 1873). Moreover, formation of one phosphate equivalent in the products to be disposed makes the work-up and purification procedures not friendly for the environment. Another major limitation related to the Corey strategy is non-stereoselective reduction of the 15-keto function in the ω-chain yielding the mixtures of 15R/15S epimers in ratios depending on reagent and conditions used. It was experimentally proved, that preparation of 15-OH single epimer with the selectivity higher than 99% is not possible following this protocol. Close similarity of physico-chemical properties of 15R and 15S epimers, hinders the removal of substantial amounts of undesired 15-epi isomer from the reaction mixture. Separation of the isomeric impurity requires implementation of laborious and multistep purification procedure to obtain the final prostaglandin analogue of high purity.

Another approach to the preparation of prostaglandin F_{2α} derivatives was proposed in some Polish patents claiming the priority of 1984, namely PL 144084 B1, PL 144085 B1, PL 149389 B1, PL 147530 B1 (EP-A-189555; U.S. Pat. No. 4,707,554) and the papers by B. Achmatowicz et al., Tetrahedron Lett. 1985, 26, 5597 and Tetrahedron 1988, 44, 4989. They first reported that addition of lithiated Corey sulfone to aldehydes could be a new method for stereospecific construction of the allylic alcohol moiety of prostaglandins.

In PL 149389 B1, the intermediates in the synthesis of prostaglandin F_{2α} analogues, eg. cloprostenol, having 13,14-en-15-one in the first attached ω-chain, were obtained in the reaction of phenylsulfonyl derivative of Corey (−)-lactole with an appropriate electrophilic epoxide. The reaction was preceded by generation of carbanion at α position to the sulphonyl group, upon treatment of the phenylsulfonyl derivative with an organometallic base or Lewis acid. The intermediate bearing β-hydroxysulfone moiety was subjected to reduction, followed by the elimination of phenylsulfonyl along with adjoining hydroxyl.

In PL 144085 B1, phenylsulfonyl derivative of Corey (−)-lactol activated with Lewis acid, was reacted with the appropriate aldehyde. Due to reductive elimination, prostaglandin F_{2α} precursors, bearing unsaturated ω-chain substituted at C-15 position with alkyl or aryl, were obtained. This approach to prostaglandin F_{2α} derivatisation does not seem superior over the method based on introduction of 13,14-en-15-one moiety under the Wittig protocol.

That strategy of construction of prostaglandin ω-chain having both hydroxyl and allyl groups requires long times, cooling to low temperatures and use of BF₃ etherate to activate bulky sulfones, making the entire process not suitable enough for industrial purposes. In addition, the troublesome stereoselective reduction of carbonyl group, followed by attachment of an α-chain is necessary, as it was already discussed above.

The growing demand for the stereoselective synthetic method to obtain prostaglandin F_{2α} analogues bearing chiral center at C-15 in the ω-chain, led us to elaborate the convergent synthesis of latanoprost, which had been described in the International Patent Application WO 2006/112742 and J. G. Martynow et al., Eur. J. Org. Chem. 2007, 689. In the described strategy, the precursor of the α-chain is attached to the Corey (−)-lactone derivative first, and then the precursor of the ω-chain possessing the asymmetric center of desired configuration at carbon atom substituted by hydroxyl. The α-chain of the (−)-Corey lactone was elongated in the reaction with [4-(4-methyl-2,6,7-trioxabicyclo[2.2.2]-1-octyl)butyl]triphenylphosphonium iodide under the Wittig protocol, and the obtained phenylsulfone, after activation with lithium N,N-bis(trimethylsilyl)amidate, was alkylated with enantiomerically pure (S)-4-phenyl-1-iodo-2-(triethylsilyloxy)butane. This method allowed to prepare 13,14-dihydro-15(R)-17-substituted-18,19,20-trinor-F_{2α} analogues, eg. latanoprost, in high diastereoisomeric excess with only trace amounts of undesired 15-epi isomer detected. Regioselective reduction and separation of regioisomeric impurities from the final products was not necessary.

The use of prostaglandins as the active substances in the ocular formulations and the requirements of drug approving authorities, compel the producers to eliminate from the end products all the impurities of potential biological activity, such as diastereoisoms of travoprost (8b-c) depicted in FIG. 3 and diastereoisoms of bimatoprost (10b-c) depicted in FIG. 4, as well as the other byproducts.

Searching for the effective process for preparation of prostaglandins having the diastereoisomeric purity, we have developed the convergent method for the synthesis of prostaglandin F_{2α} analogues, especially travoprost and bimatoprost, starting from the structurally advanced prostaglandin phenylsulfones represented by the formula (II), disclosed in WO 2006/112742.

### SUMMARY OF THE INVENTION

The present invention relates to the process for preparation of prostaglandin F_{2α} analogues bearing 13,14-en-15-ol ω-chain having an 15R or 15S optical configuration at stereogenic center, represented by the general formula (I),

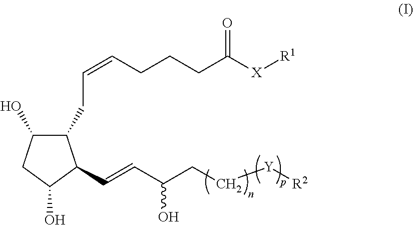

wherein:

X represents —O— or —NH—;

R¹ is H or C₁₋₃-alkyl;

Y represents —O—;

R² is H or phenyl group unsubstituted or substituted by trifluoromethyl group;

n represents an integer 0 or 1;

p represents an integer 0 or 1,

the process comprising the steps of:
(a) treatment of phenylsulfone of the formula (II)

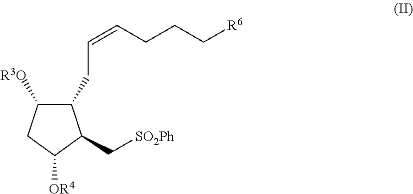

wherein
R³ and R⁴ independently represent hydroxyl protecting group —Si(R⁹)(R¹⁰)(R¹¹), where R⁹-R¹¹ are the same or different and are C₁₋₆-alkyl or phenyl;
R⁶ is the orthoester group, represented by the general formula (III)

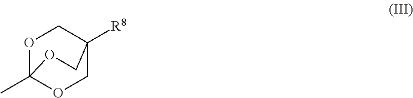

wherein
R⁸ is H or C₁-C₆-alkyl,
or
R⁶ represents —C(OR¹²)₃ orthoester group, wherein R¹² is C₁-C₆-alkyl;
with a strong organometallic base, generating the α-sulfonyl carboanion of the compound (II),
(b) addition of the α-sulfonyl carbanion in situ to aldehyde having the optical configuration at stereogenic center corresponding to 15R or 15S optical configuration of the target prostaglandin, respectively, represented by the formula (IV),

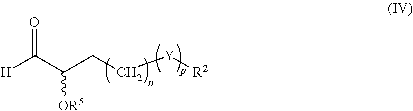

wherein
R⁵ represents hydroxyl protecting group —Si(R⁹)(R¹⁰)(R¹¹), where R⁹-R¹¹ are the same or different and represent C₁₋₆-alkyl or phenyl;
and
Y, R², n and p have the same meaning as defined for the compound (I), to yield the mixture of diastereoisomers of 3-hydroxysulfones of the general formula (V):

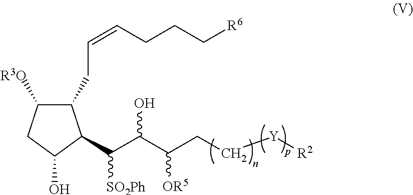

wherein R²-R⁶, Y, n and p have the same meaning as defied for the compound (1),
(c) reductive desulfonation of the mixture of j-hydroxysulfones of the general formula (V), to yield the compound having the 15R or 15S optical configuration, represented by the formula (VI):

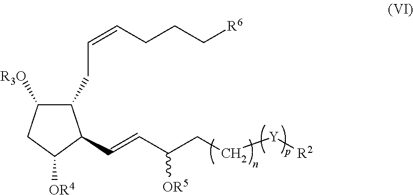

wherein R²-R⁶, Y, n and p have the same meaning as defined for the compound (I)
(d) removing R³, R⁴, R⁵ hydroxyl protecting groups to yield the compound having the 15R or 15S optical configuration, represented by the formula (VII):

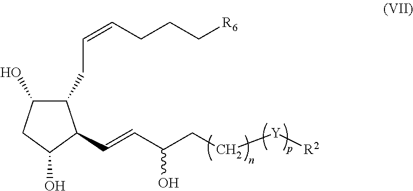

wherein
R², R⁶, Y, n and p have the same meaning as defined for the compound (I),
(e) hydrolysis of the compound of formula (VII) under acidic conditions, to yield the product having the 15R or 15S optical configuration, represented by the formula (VIII):

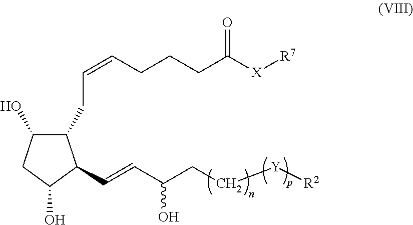

wherein
X represents —O—;
R⁷ represents —CH₂—C(CH₂OH)₂—R⁸ or R¹² respectively;
wherein R⁸ is H or C₁-C₆-alkyl and R¹² is C₁-C₆-alkyl;
R², Y, n and p have the same meaning as defied for the compound (I),
(f) hydrolysis of the compound of formula (VIII) under basic conditions, to yield the compound having the 15R or 15S optical configuration, represented by the formula (IA):

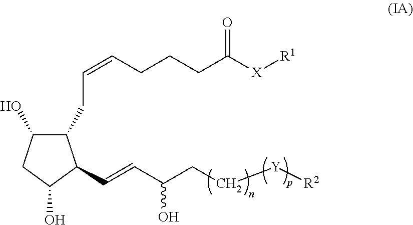

wherein
X represents —O—;
R¹ is H;
R², Y, n and p have the same meaning as defied for the compound (I),
and then
alkylating the compound of formula (IA) with C₁₋₃-alkyl halogen in the presence of strong base, to obtain the compound having the 15R or 15S optical configuration, represented by the formula (IB):

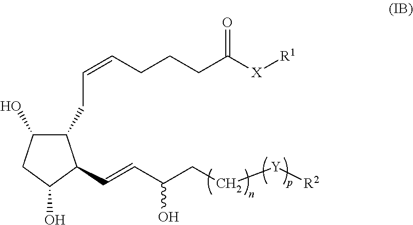

wherein
X represents —O—;
R¹ is C₁₋₃-alkyl; and
R², Y, n and p have the same meaning as defied for the compound (I),

and, optionally,
reacting the compound of formula (IB) with the amine of the formula (IX)

R¹NH₂ (IX)
wherein R¹ is C₁₋₃-alkyl,
to obtain prostamid having the 15R or 15S optical configuration, represented by the formula (IC):

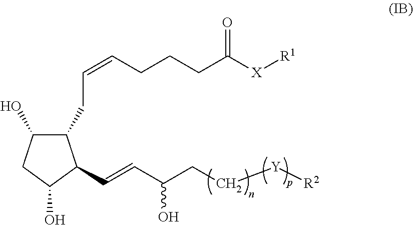

wherein
X represents —NH—
R¹ is C₁₋₃-alkyl;
R², Y, n and p have the same meaning as defined for the compound (I);

or, optionally.
reacting the compound of formula (VIII) with amine of the formula (IX)

R¹NH₂ (IX)
wherein R¹ is C₁₋₃-alkyl,
to obtain prostamid having the 15R or 15S optical configuration represented by the formula (IC)

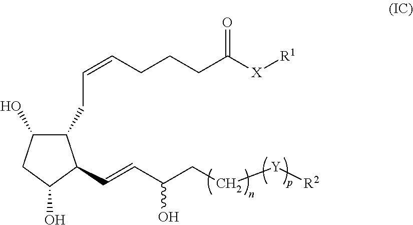

wherein
X represents —NH—
R¹ is C₁₋₃-alkyl;
R², Y, n and p have the same meaning as defined for the compound (I).

The invention provides also the new intermediates in the synthesis of prostaglandin F_{2α} analogues, which are β-hydroxysulfones having the 15R or 15S optical configuration, represented by the formula (V):

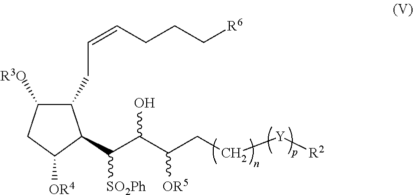

wherein
R³, R⁴ and R⁶ independently represent hydroxyl protecting groups —Si(R⁹)(R¹⁰)(R¹¹), wherein R⁹-R¹¹ are the same or different and are C₁₋₆-alkyl or phenyl;
R⁶ is an orthoester group, represented by the general formula (III):

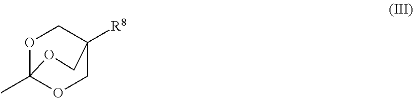

wherein
R⁸ is H or C₁-C₆-alkyl,
or
R⁶ represents —C(OR¹²)₃ orthoester group, wherein R² is C₁-C₆-alkyl;
Y represents —O—;
R² is H or phenyl unsubstitute or substituted by trifluoromethyl;
n represents an integer 0 or 1;
p represents an integer 0 or 1.

Preferably, in the compound of the formula (V):
R³, R⁴ and R⁵ independently represent —Si(R⁹)(R¹⁰)(R¹¹) silyl hydroxyl protecting groups, wherein R⁹-R¹¹ are the same or different and are C₁₋₆-alkyl or phenyl;
R⁶ is an orthoester group, represented by the general formula (III),

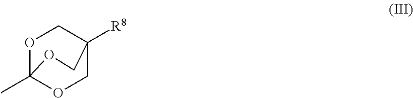

wherein
R⁸ is H or C₁-C₆-alkyl,
and
when Y represents —O— and p=1, than R² is phenyl substituted in meta position by trifluoromethyl, and n=0;
and when Y represents —CH₂— and p=0, than R² is phenyl, and n=1.

Another aspect of the invention are the intermediates in the synthesis of prostaglandin F_{2α} analogues, having the 15R or 15S optical configuration, represented by the formula (VI)

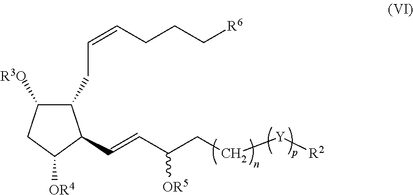

wherein
R³, R⁴ and R⁵ independently represent —Si(R⁹)(R¹⁰)(R¹¹) silyl hydroxyl protecting groups;
R⁶ is an orthoester group, represented by the general formula (III),

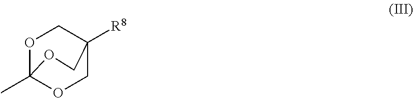

wherein
R⁸ is H or C₁-C₆-alkyl,
or
R⁶ represents —C(OR¹²)₃ orthoester group, wherein R¹² is C₁-C₆-alkyl;
Y represents —O—;
R² is H or phenyl unsubstituted or substituted by trifluoromethyl;
n represents an integer 0 or 1;
p represents an integer 0 or 1.

Preferably, in the compound of the formula (VI):
R³, R⁴ and R⁵ independently represent hydroxyl protecting groups —Si(R⁹)(R¹⁰)(R¹¹), wherein R₉-R₁₁ are the same or different and represent C₁₋₆-alkyl or phenyl;
R⁶ is an orthoester group, represented by the general formula (III),

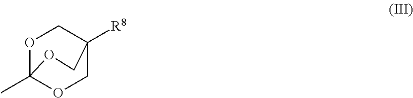

wherein
R⁸ is H or C₁₋₆-alkyl;
and
when Y represents —O— and p=1, than R² is phenyl substituted in meta position by trifluoromethyl, and n=0;
and when Y is —CH₂— and p=0, than R² is phenyl, and n=1.

Another aspect of the invention are the intermediates in the synthesis of prostaglandin F_{2α} analogues having the 15R or 15S optical configuration, represented by the formula (VII)

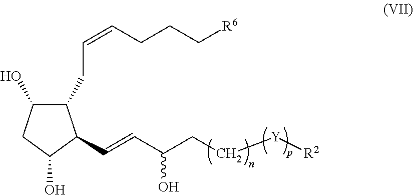

wherein
R⁷ represents —CH₂—C(CH₂OH)₂—R⁸ group, wherein R⁸ is H or C₁-C₆-alkyl,
or
R⁷ is —C(OR¹²)₃ orthoester group, wherein R¹² is C₁-C₆-alkyl;
Y represents —O—;
R² is H or phenyl unsubstituted or substituted by trifluoromethyl;
n represents an integer 0 or 1;
p represents an integer 0 or 1.

Preferably, in the compound of the formula (VII):
R⁷ represents —CH₂—C(CH₂OH)₂—R⁸ group, wherein R⁸ is H or C₁-C₆-alkyl,
Y represents —O—;
and
when Y represents —O— and p=1, than R² is phenyl substituted in meta position by trifluoromethyl, and n=0;
and when Y is —CH₂— and p=0, than R² is phenyl, and n=1.

In the further aspect, the invention provides the intermediates in the synthesis of prostaglandin F_{2α}, analogues having the 15R or 15S optical configuration, represented by the formula (VIII)

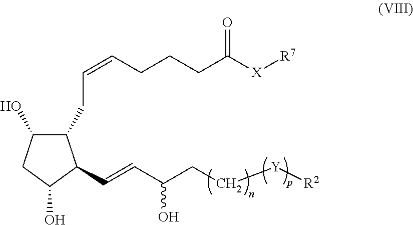

wherein
X represents —O—;
R⁷ represents —CH₂—C(CH₂OH)₂—R⁸ or R¹²,
wherein R⁸ is H or C₁-C₆-alkyl, and R¹² is C₁-C₆-alkyl;
Y represents —O—;
R² is H or phenyl unsubstituted or substituted by trifluoromethyl;
n represents an integer 0 or 1;
p represents an integer 0 or 1.

The preferred compound of general formula (VIII) has the formula (VIIIA):

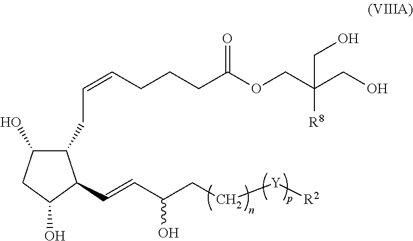

wherein
when Y represents —O— and p=1, than R² is phenyl substituted in meta position by trifluoromethyl, and n=0;
and when Y represents —CH₂— and p=0, than R² is phenyl, and n=1.

In the further aspect, the invention provides the aldehyde synthons represented by formula (IV) having an S or R optical configuration at stereogenic center. These compounds have not yet been reported to have been obtained in the optically active form.

In the preferred embodiment of that aspect of the invention, the new aldehyde synthons have the formula (IV), wherein: when Y is —O— and p=1, then R² is phenyl substituted in the meta position by trifluoromethyl and n=0; or when Y is —CH₂— and p=0, then R² is phenyl and n=1.

Especially preferred aldehyde synthons are: (S)-(−)-2-hydroxy-3-(3-fluoromethylphenoxyl)propanal and its derivative with the protected hydroxyl group, ie. (S)-(−)-2-(tert-butyldimethylsilyloxy)-3-(3-trifluoromethylphenoxy)propanal, which are useful starting compounds for ω chain building in the synthesis of fluprostenol and travoprost. They have the stereogenic center at C-2 carbon atom corresponding to C-15 of a target prostaglandin, and have the chirality corresponding to the configuration 15S of fluprostenol and travoprost.

The other preferred aldehyde synthons are: (S)-(−)-2-hydroxy-4-phenylbutanal as well as its derivative with the protected hydroxyl group, ie. (S)-(−)-2-(tert-butyldimethylsililoxy)-4-phenylbutanal, which are useful starting compounds for ω chain building in the synthesis of bimatoprost. They have the stereogenic center at C-2 carbon atom corresponding to C-15 of a target prostaglandin, and have the chirality corresponding to the configuration 15S of bimatoprostu.

Thus, the preferred aldehyde synthons of formula (IV) having the 2S or 2R optical configuration at the stereogenic center, that are useful in the process for preparation of prostaglandin F_{2α} analogues and their epimers under Julia-Lithgoe protocol, are selected from the group comprising:
(S)-(−)-2-(tert-butyldimethylsililoxy)-3-(3-trifluoromethylphenoxy)propanal,
(R)-(+)-2-(tert-butyldimethylsililxsy)-3-(3-trifluoromethylphenoxy)propanal,
(S)-(−)-2-(tert-butyldimethylsililoxy)-4-phenylbutanal,
(R)-(+)-2-(tert-butyldimetylosililoksy)-4-fenylobutanal.

The aldehyde synthons of formula (IV) having the S or R optical configuration at the stereogenic center possessing a very high enantiomeric purity above 99% ee, especially above 99.5% ee, are conveniently obtainable by the method according to the invention, depicted in the Scheme 5.

The process for preparation of aldehyde synthones having the optical configuration S or R at the stereogenic center and having the enantiomeric excess greater than 99% ee, represented by the formula (IV),

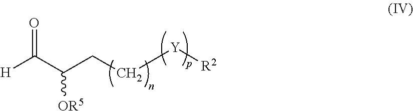

wherein
Y represents —O—;
R² is H or phenyl group unsubstituted or substituted by trifluoromethyl group;
n represents an integer 0 or 1;
p represents an integer 0 or 1;
R⁵ represents hydroxyl protecting group —Si(R⁹)(R¹⁰)(R¹¹), and R⁹-R¹¹ are the same or different and represent C₁₋₆-alkyl or phenyl, is characterized in that:
(a) the primary hydoxyl groups of 1,2-diol of configuration at stereogenic center 2S or 2R having the enantiomeric excess greater than 99% ee, represented by the formula (IV-1)

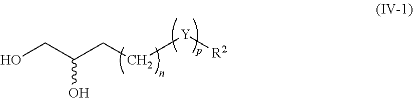

wherein R², Y, n and p have the meaning as defined for formula (IV), are selectively esterificated with pivaloyl chloride under basic conditions, to obtain α-hydroxypivaloate of formula (IV-2)

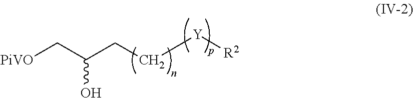

wherein R², Y, n and p have the meaning as defined for formula (IV),
(b) the secondary hydroxyl group of α-hydroxypivaloate of formula (IV-2) is protected by silylation with silyl chloride of formula R⁵Cl,
wherein
   R⁵ represents —Si(R⁹)(R¹⁰)(R¹¹), where R⁹-R¹¹ are the same or different and represent C₁₋₆-alkyl or phenyl,
   to obtain the compound of formula (IV-3),

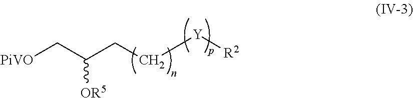

wherein
   R⁵ represents hydroxyl protecting group —Si(R)(R¹⁰)(R¹¹), and R⁹-R¹¹ are the same or different and represent C₁₋₆-alkyl or phenyl, and
   R², Y, n and p have the meaning as defined for formula (IV);
(c) the pivaloate ester of formula (IV-3) is deprotected with diisobutylaluminum hydride, to obtain the alcohol of formula (IV-4)

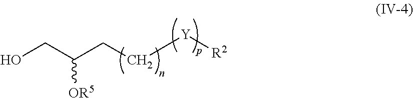

wherein
   R⁵ represents hydroxyl protecting group —Si(R⁹)(R¹⁰)(R¹¹), and R⁹-R¹¹ are the same or different and represent C₁₋₆-alkyl or phenyl, and
   R², Y, n and p have the meaning as defined for formula (IV),
(d) the alcohol of formula (IV-4) is oxidized to the corresponding α,β-unsaturated aldehyde represented by formula (IV-5)

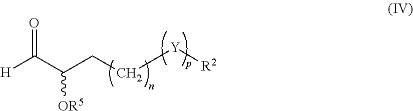

wherein
   R⁵ represents hydroxyl protecting group —Si(R⁹)(R¹⁰)(R¹¹), and R⁹-R¹¹ are the same or different and represent C₁₄-alkyl or phenyl, and
   R², Y, n and p have the meaning as defined for formula (IV),
and, optionally
(e) the protecting groups R⁵ are removed to give the aldehyde of formula (IVA)

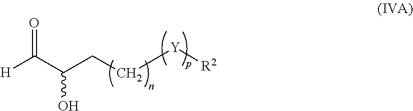

wherein R², Y, n and p have the meaning as defined for formula (IV).

### BRIEF DESCRIPTION OF THE FIGURES

FIG. 1 depicts Scheme 1, which illustrates the synthetic route of synthesis of travoprost.

FIG. 2 depicts Scheme 2, which illustrates the synthetic route of synthesis of bimatoprost.

FIG. 3 presents the main travoprost impurities.

FIG. 4 presents the main bimatoprost impurities.

FIG. 5 depicts Scheme 3, which illustrates the synthetic route of synthesis of aldehyde synthons of formula (IV).

FIG. 6 depicts Scheme 4, which illustrates the synthetic route of synthesis of aldehyde synthon for the synthesis of travoprost.

FIG. 7 depicts Scheme 5, which illustrates the synthetic route of synthesis of aldehyde synthon for the synthesis of bimatoprost.

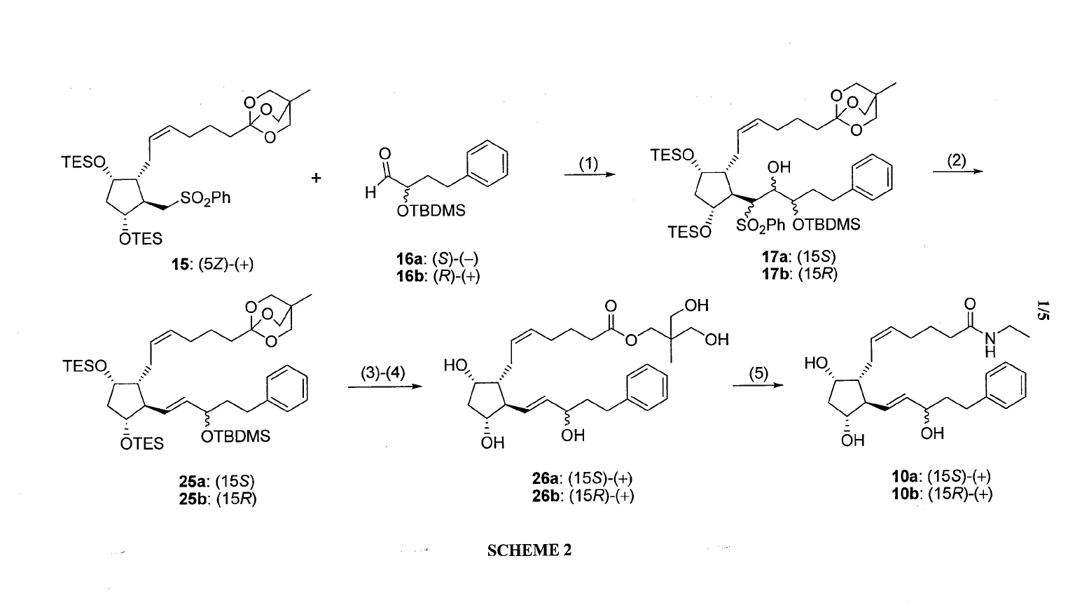

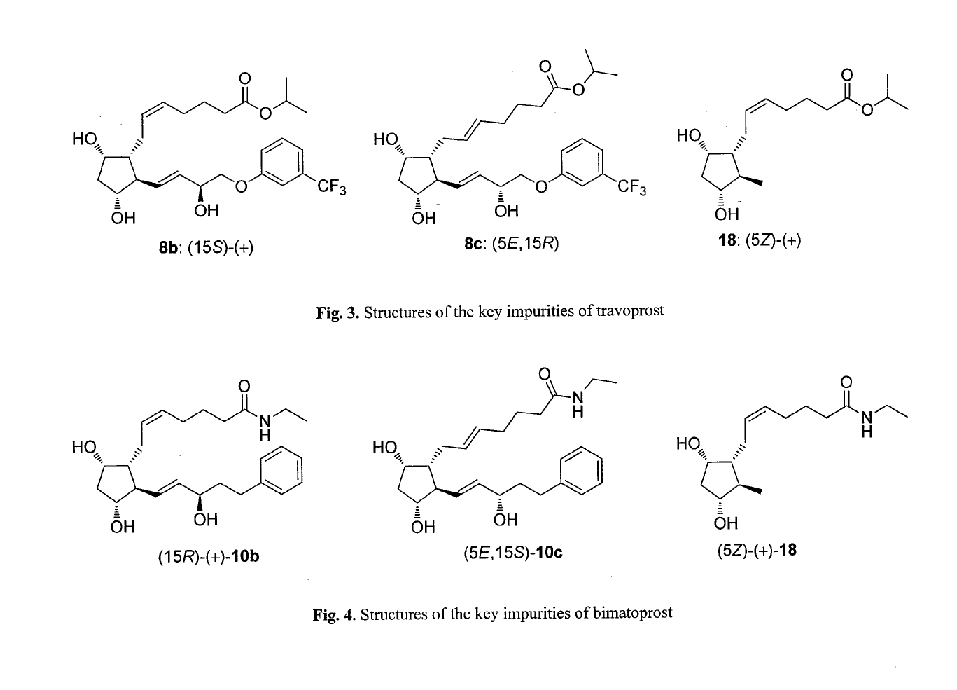

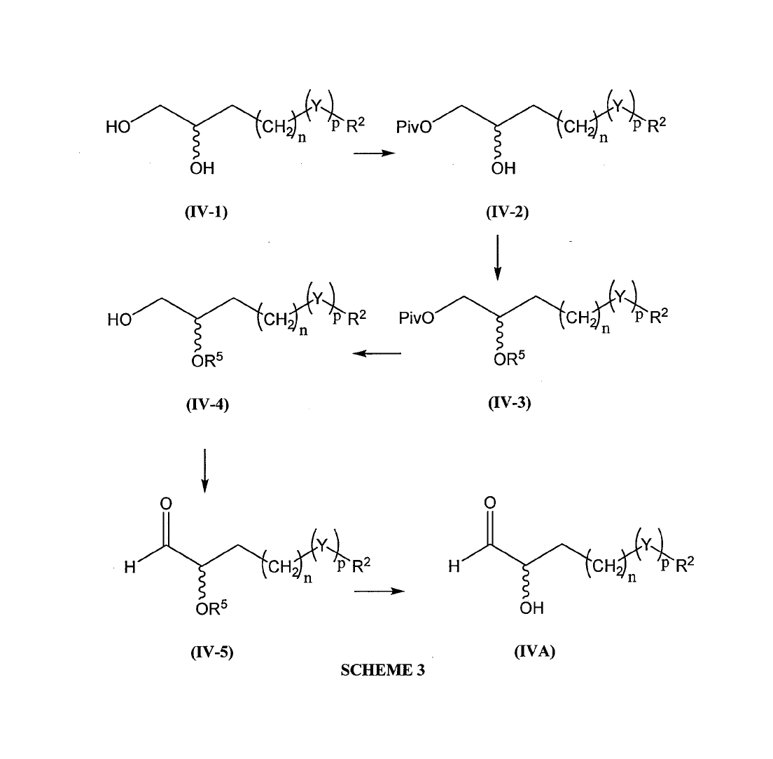

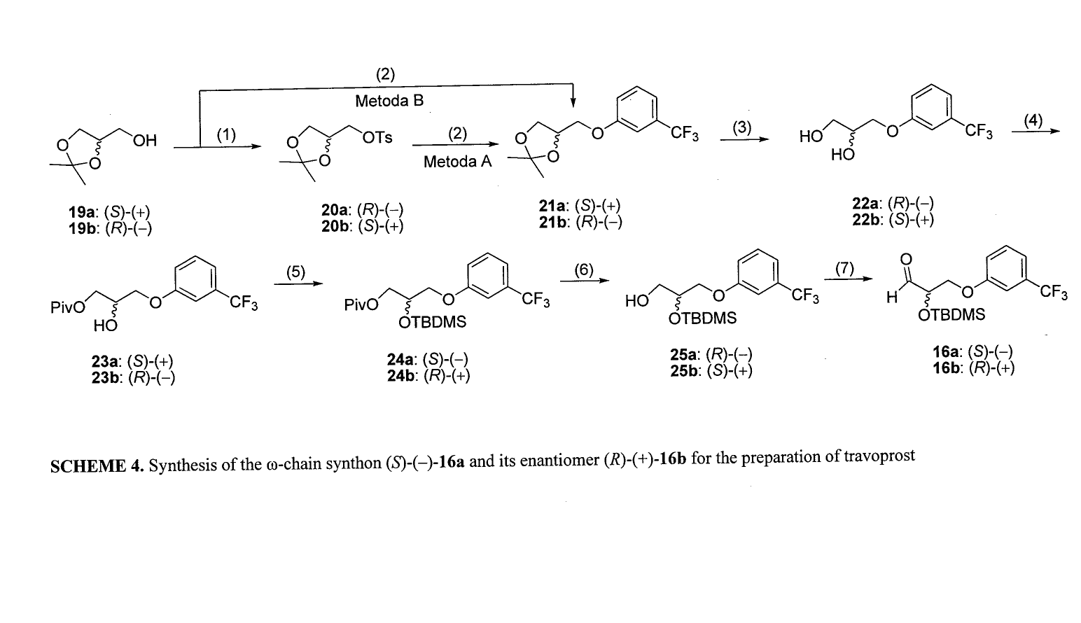

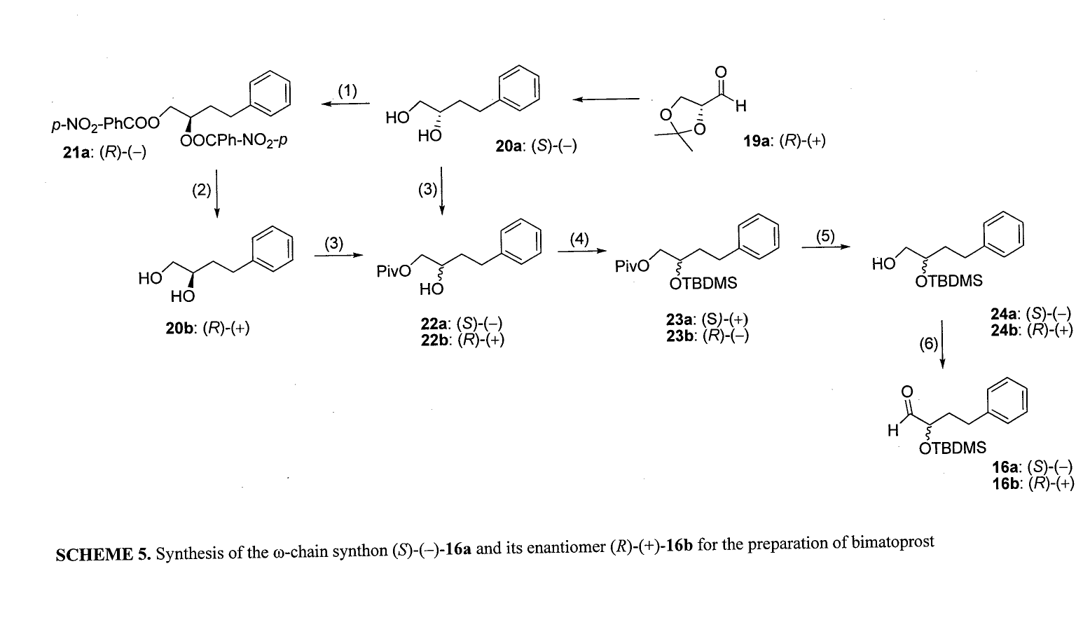

### DETAILED DESCRIPTION OF THE INVENTION

Convergent synthesis approach providing prostaglandin F_{2α} analogues represented by the general formula (I), having different substituents in a and co chains, is accomplished by using the structurally advanced prostaglandin sulfones of the formula (II) in Julia-Lythgoe olefination. This strategy adopted in the present invention was revealed in WO 2006/112742. Julia-Lythgoe olefination reaction, described in the following publications: M. Julia, J-M. Paris, Tetrahedron Lett. 1973, 14, 4833; P. J. Kocieński, B. Lythgoe, S. Ruston, J. Chem. Soc. Perkin Trans. 1 1978, 19, 829; P. J. Kocieński, B. Lythgoe, I. Waterhouse, J. Chem. Soc. Perkin Trans. 1 1980, 1045; P. J. Kocieński, Phosphorus Sulphur 1985, 24, 97; P. R. Blakemore, W. J. Cole, P. J. Kocieński, A. Morley, Synlett. 1998, 26, is a several-step process, embracing the key step of phenylsulfonyl carbanion addition to aldehyde, which results in (E)-alkene formation.

There are plethora of examples concerning (E)-alkene preparations under Julia-Lythgoe olefination protocol, but this method has not been used yet for ω-chain elongation of prostaglandins having bulky substitutent at α position. Moderate reactivity of the aldehyde (IV), which is the precursor of ω-chain, as well as low stability of the protecting groups under reaction conditions, were the main obstacles to obtain phenylsulfone of the formula (I) with the ω-chain elongated.

The authors of the present invention solved these synthetic problems, choosing appropriate base as well as using properly protected hydroxyaldehyde (IV) to perform chain elongation of phenylsulfonyl (II), in an efficient and diastereoselective way.

According to the present invention, addition of phenylsulfonyl carbanion to hydroxyaldehyde, followed by subsequent reductive elimination of β-hydroxysulfones enable incorporation of the allyl moiety into ω-chain of the target prostaglandin. The reaction proceeds with unique stereoselectivity, furnishing the formation of relative cis/trans configuration of α- and ω-side chains and trans configuration of C-13/C-14 double bond in ω-chain. The mixture of diastereoisomers of hydroxysulfones can be converted into desired prostaglandin F_{2α} of the formula (I), for example travoprost or bimatoprost, in a few synthetic steps, as it is depicted in the Scheme 1.

In accordance with the present strategy, R or S configuration at the stereogenic center (carbon atom substituted by hydroxyl group) of the aldehyde of the formula (IV), which is the synthon of ω-chain, corresponds to the configuration of the final prostaglandin derivative of the formula (I). Preferably, the aldehyde (IV) having the stereogenic center with 2S configuration, which corresponds to 15R configuration in travoprost and fluprostenol or 15S configuration in bimatoprost, is used.

The aldehyde of the formula (IV) should posses the high enantiomeric excess. The definition of enantiomeric excess is included in the monograph: E. L. Eliel and al., “Stereochemistry of Organic Compounds” John Wiley and Sons, Inc., New York, N.Y., 1994. Use of the aldehyde (IV) of enantiomeric excess above 99%, preferably higher than 99.5%, yields the final product (I) of the demanded high diastereoisomeric purity.

The required enantiomeric purity of the aldehyde synthons of formula (IV) may be achieved due to the use of the 1,2-diols of formula (IV-1) possessing the enantiomeric excess above 99% ee, especially above 99.5% ee, for their synthesis.

The optically active 1,2-diols are commercially available or can be easily prepared from the available reagents using the methods known in the art.

The glicerol derivatives, including enantiomeric (R)-(−)-3-trifluoromethylphenoxy-1,2-propanediol, has been described by W. L. Nelson et al., J. Org. Chem. 1977, 42, 1006.

(S)-(−)-4-Phenyl-1,2-butanediol has been disclosed in J. G. Martynow et al., Eur. J. Org. Chem. 2007, 689.

The synthesis of (2R)-1,2-dihydroxy-4-phenylbutan possessing the enantiomeric excess 84% ee, with the use of (DHQD)₂PHAL as the catalyst, has been described in, eg., Z.-M. Wang at al., Tetrahedron Lett. 34 (1993), 2267-2270.

Optically active phenyl-substituted 1,2-diols as well as 2,3-O-izopropylidene-D-gliceric aldehyde used for the synthesis of 1,2-diols can be conveniently prepared using the protocol described in C. R. Schmid et al., Organic Syntheses, Coll. Vol. 9 (1998), 450, from the commercially available 1,2:5,6-di-O-izopropylidene-D-mannitol. It is first converted ino bis-acetonide, and then into 2,3-O-izopropylidene-D-gliceric aldehyde under sodium periodate treatment.

The process for preparation of phenyl-substituted and trifluoromethylphenoxy-substituted 1,2-diols is depicted, by the way of example in Schemes 4 and 5. Scheme 4 illustrates the process for preparation of optically active aldehyde synthons: (S)-(−)-2-(tert-butyldimethylsililoxy)-3-(3-trifluoromethylphenoxy)propanal (1a) and its epimer (R)-(+)-2-(tert-butyldimethylsililoxy)-3-(3-trifluoromethylphenoxy)propanal (1 b). Scheme 5 illustrates the process for preparation of optically active aldehyde synthons: (S)-(−)-2-(tert-butyldimethylsililoxy)-4-phenylbutanal (9a) and its epimer (R)-(+)-2-(tert-butylodimethylsililoxy)-4-phenylbutanal (9b).

According to Scheme 4, the acetonide (21a) is synthesized in high enantiomeric purity from solketal (19a), which is commercially available or can be easily prepared from 1,2:5,6-di-O-isopropylidene-D-mannitol. The solketal (19a) is first converted into the known tosylate (R)-(−)-20a with p-toluenesulfonyl chloride in pyridine according to the standard procedures. O-Alkylation of 3-trifluoromethylphenol with tosylate (20a) gives the acetonide (21a). The enantiomeric acetonide (R)-(−)-21b is synthesized by the same synthetic route as illustrated on Scheme 3, but using the solketal (R)-(−)-19b as a starting material. An alternative one-step approach to the preparation of (S)-(+)-21a could be etherification of solketal (19a) with 3-trifluoromethylphenol under Mitsunobu reaction conditions. The acetonide group in aryl ether (21a) is removed by using aqueous hydrochloric acid in acetone to afford the diol (R)-(−)-22a.

According to the Scheme 5, the aldehyde synthon (16a) or its enantiomeric impurity (16b) could be prepared in a four-step syntheses from optically active diol (S)-(−)-20a or (R)-(+)-20a. The desired enantiomeric purity of the aldehyde (16a) is achieved by employing the known (R)-(+)-2,2-dimethyl-1,3-dioxolane-4-carboxaldehyde (19) as a source of chirality. The diol (21a) is synthesized in high enantiomeric purity from the aldehyde (19). The enantiomeric impurity (R)-(+)-20b is synthesized by Mitsunobu esterification of the diol (20a) with the excess of p-nitrobenzoic acid to the corresponding bis(4-nitrobenzoate) (21a). Hydrolysis of diester (21a) under mild basic conditions gives the diol (R)-(+)-20b.

The other optically active 1,2-diols being the substrates for the preparation of the aldehyde synthones of formula (IV) could be prepared by the same procedure.

The critical factor in the synthesis of aldehydes of formula (IV) is the strategy of selecting protecting groups for hydroxyl groups in the diols, that would minimize side reactions. In the present process, the most advantageous is a selective esterification of the primary hydroxyl group of 1,2-diol with pivaloyl chloride to afford the pivaloate α-hydroxy ester as the main product of the reaction, while the secondary hydroxyl group is protected by silylation with silyl chlorides to give the silyl ether.

The selection of silyl derivatives as protecting groups results from few factors such as, their stability under varying reaction conditions, their bulkiness, enhancement of the chemical reactivity of the molecule and easy removal in the last step of the prostaglandins synthesis. The selected silyl protecting groups are for example trialkylsilyl, dialkylarylsilyl, alkyldiarylsilyl or triarylsilyl groups, represented by the formula —Si(R⁹)(R¹⁰)(R¹¹), wherein R⁹-R¹¹ are the same or different and are C₁₋₆-alkyl or phenyl. Preferably, selected silyl groups are trimethylsilyl, trethylsilyl, tert-butyldimethylsilyl, tert-butyldiphenylsilyl, triphenylsilyl groups.

Preferably, tert-butyldimethylsilyl group (TBDMS) is used as R⁵ hydroxyl protecting group in the aldehyde (IV). TBDMS group affects electronoaceptor balance of the molecule, thus increasing aldehyde reactivity during nucleophilic substitution, its moderate size does not cause steric hindrance which may hamper the reaction progress. Moreover, tert-butyldimethylsilyl protecting group provides better 15R/15S stereoselectivity for ω-chain elongation than triethylsilyl protection.

The selective esterification of the primary hydroxyl group of 1,2-diol (IV-1) with pivaloyl chloride under basic conditions, eg. in pyridine, affords the pivaloate α-hydroxy esters (IV-2) possessing the desired optical configuration as the main products of this reaction.

The subsequent step involves the deprotection of the C-1 pivaloate ester (IV-2). The pivaloyl group is removed from the hydroxyl at the position a in relation to the sililated secondary hydroxyl group by means of reduction, preferably with diisobutylaluminum hydride (DIBAL-H), providing the primary alcohol (IV-3) having the desired optical configuration and the high enantiomeric purity.

Then, the alcohol (IV-3) is oxidized to the aldehyde (IV-4).

There are plenty of methods to obtain aldehydes from alcohols, like the Parikh-Doering oxidation with DMSO and SO₃/pyridine in the presence of organic base (SO₃.Py, DMSO, Et₃N), Swern method with oxalil chloride in DMSO in the presence of organic base, the oxidation with pyridinium chlorochromate (DCC) or Dess-Martin periodinane.

In case of optically active compounds of the invention, most of the above mentioned methods appears to be not selective enough or generate the undesired by-products.

For example, in case of the alcohols (IV-3) with 3-trifluoromethylphenoxyl substituent, the Parikh-Doering oxidation results in the aldehyde formation immediately followed by 3-trifluoromethylphenol elimination to the more stable α,β-unsaturated aldehyde, eg. 2-(tert-butyldimethylsililoxy)propenal.

The attempts to synthesize the aldehydes by pyridinium chlorochromate oxidation also failed, mainly due to the low reactivity of the alcohol (IV-3), accompanied by decomposition of the product under long-term oxidation conditions.

Thus, in the present invention, oxidation of the alcohol (IV-3) with Dess-Martin periodinane (P. R. Blakemore et al., Org. Biomol. Chem. 2005, 3, 1365) affords the crude aldehyde (IV-4) with good yield and high chemical purity. The use of Dess-Martin periodinane allows to avoid the byproducts formation, avoiding long reaction times, difficult workup procedures or the need to apply a large excess of the oxidizing agent, and first of all racemization of the chiral centers of optically active compounds. Thus, the enantiomeric purity of the aldehyde ω-chain synthon (IV-4) is the same as the starting alcohol (IV-3).

The compounds of formula (IV) obtained in such manner, are characterized by high enantiomeric purity, demanded in the Julia-Lythgoe reaction, and therefore can be used in the synthesis of prostaglandin analogues, as it is described in the invention.

The groups chosen for the protection of compounds (II) and (IV) may be the same or different.

The selected hydroxyl protecting groups of the phenylsulfone (II), R³ and R⁴, are for example triethylsilyl groups (TES).

The phenylsulfone (II) used as the starting material, is the precursor of carboxyl, ester or amide group in α chain of the target prostaglandin F_{2α}, R⁶ group is an orthoester or oxabicyclo[2.2.2]octan (OBO) group.

Application of an orthoester or oxabicyclo[2.2.2]octan masking carboxyl group has been comprehensively discussed in the literature, for instance: T. W. Greene, P. G. M. Wuts “Protective Groups in Organic Synthesis”, ed. 3, John Wiley and Sons, Inc., New York, N.Y., 1999; chapter V, and in U. Pindur; J. Mueller, C. Flo, H. Witzell Chem. Soc. Rev. 1987, 75. Only few examples, describing oxabicycloocane group application in the synthesis of prostaglandin can be found (G. H. Verdoorn et al. South African Journal of Chemistry 40 (1987), 134-8; E. J. Corey, X.-M. Cheng “The Logic of Chemical Synthesis” John Wiley and Sons, Inc., New York, N.Y., 1989; rozdzial XI). The seldom use of this protecting group effects from moderate stability of oxabicycloocane under acidic conditions. The compounds of the 4-methyl-2,6,7-trioxabicyclo[2.2.2]octan structure are easily hydrolyzed to corresponding 2,2-bis(hydroxymethyl)-1-propyl esters, which can be in turn converted into other esters, like for example alkyl esters, salts or carboxylic acids (P. J. Kocienski “Protecting Groups”, Georg Thieme Verlag, Stuttgart, 1994; T. W. Greene, P. G. M. Wuts “Protective Groups in Organic Synthesis”, ed. 3, John Wiley and Sons, Inc., New York, N.Y., 1999; J. March “Advanced Organic Chemistry” John Wiley and Sons, New York, N.Y., 1992). Under carefully selected conditions, 4-methyl-2,6,7-trioxabicyclo[2.2.2]octan groups as well as the other orthoesters can be used successfully as carboxyl protecting groups, especially in the presence of a base.

The crucial step In the Julia-Lythgoe olefination process according to the present invention is nucleophilic addition of the α-sulfonyl (II) carbanion to the aldehyde (IV).

The α-sulfonyl carbanion is generated from substituted phenylsulfone (II) in the presence of a strong organometallic bases. The formation of stabilized —CH⁻—SO₂—Ar carbanions due to activation of (arylsulfonyl)methylene groups under basic conditions, has been already revealed in the scientific literature, See: P. E. Magnus, Tetrahedron 33 (1977), 2019; B. M. Trost, Bull. Chem. Soc. Jpn. 61 (1988), 107; N. S. Simpkins, Tetrahedron 46 (1990), 6951. Among bases, which were used to generate carbanions; n-butyllithium, potassium tert-butanolate, lithium or potassium heksamethyldisilazyde, lithium diisopropylamide or lithium bis(trimethylsilyl)amide Me₃-Si—N(Li)-Si-Me₃ had been described in I. R. Baldwin, R. J. Whitby Chem. Commun. (2003), 2786-2787.

According to the present invention, sulfonyl carbanion is generated in situ from the phenylsulfone of the formula (II) using alkali metal amide as a base, in a polar, aprotic solvent such as tetrahydrofuran. Preferably the base is selected from the group comprising lithium N,N-bis(trimethylsilyl)amide, sodium N,N-bis(trimethylsilyl)amide, lithium diisopropylamide and sodium diisopropylamide.

More preferably, lithium diisopropylamide is used as the base, resulting in high yield and high stereoselectivity of the reaction.

Nucleophilic addition of phenylsulfon (II) carbanion to the aldehyde (IV) results in the formation of diastereoisomeric mixture of β-hydroxysulfones represented by the general formula (V),

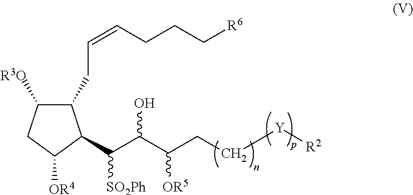

wherein R²-R⁶, Y, n and p have the same meaning as defined before.

In the next step (c), diastereoisomeric mixture of β-hydroxysulfones of the formula (V) is subjected to reductive elimination, furnishing formation of the compound represented by the general formula (VI):

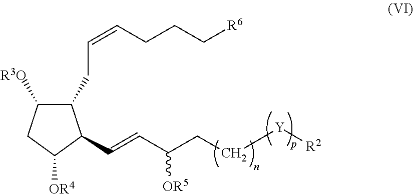

wherein R²-R⁶, Y, n and p have the same meaning as defined before.

In general, removal of arylsulfonyl group from the substituted (arylsulfonyl)alkanes can be accomplished by reductive elimination, carried out under different conditions, depending on the structure of the compound (Y. Liu, Y. Zhang, Org. Prep. Proc. Int. 33 (2001), 372). Metals dissolved in liquid ammonia (np. J. R. Hwu at al., J. Org. Chem. 61 (1996), 1493-1499); reduction with Mg/MeOH or Mg/EtOH+HgCl₂ (G. H. Lee at al., Tetrahedron Lett. 34 (1993), 4541-2; A. C. Brown, L. A. Carpino, J. Org. Chem. 50 (1985), 1749-50) and sodium amalgam in Na₂HPO₄ buffered MeOH solution (B. M. Trost at al., Tetrahedron Lett. 17 (1976), 3477-8) can be mentioned among general reductive methods. Reductive desulfonylation can be accompanied by the formation of alkene by-products, which are the products of ArS(O)OH group elimination (B. M. Trost at al., Tetrahedron Lett. 17 (1976), 3477-8).

Preferably, reductive elimination is performed using sodium amalgam (Na/Hg) in Na₂HPO₄ buffered medium.

In the next step, silyl group is removed from the crude orthoester (VI) using, for example, hydrogen fluoride or tetra-n-butylammonium fluoride, yielding the compound of formula (VII)

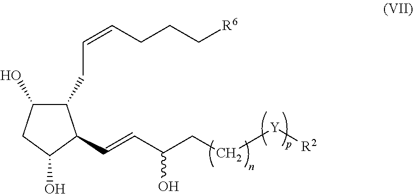

wherein
R², R⁶, Y, n and p have the same meaning as defined for the compound (I).

In the following step, R⁶ an orthoester group of the compound of the formula (VII) is hydrolyzed in the aqueous solution of weak acid, preferably, organic acid, for example tartaric, oxalic or citric acid, to yield the compound of the formula (VIII)

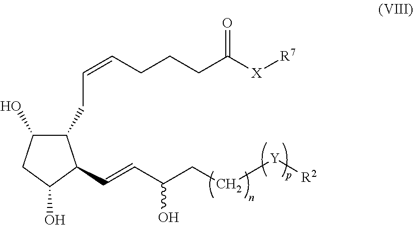

wherein
X represents —O—;
R⁷ represents —CH₂—C(CH₂OH)₂—R⁸ or R¹² group respectively,
wherein R⁸ is H or C₁-C₆-alkyl, and R¹² is C₁-C₆-alkyl;
Y represents —O—;
R² is H or phenyl unsubstituted or substituted by trifluoromethyl;
n represents an integer 0 or 1;
p represents an integer 0 or 1.

When starting sulfone of the formula (II) is possessing R⁶ group of the formula (III) which is an —OBO—R⁸, than the compound (VIII) is represented by the formula (VIIIA)

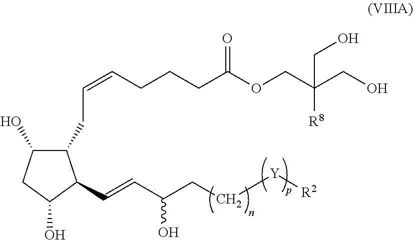

wherein R², Y, n and p have the same meaning as defined for the compound (I), and R⁸ is C₁₋₆-alkyl.

When R⁶ group of the starting sulfone of the formula (II) is an —C(OR¹²)₃ orthoester group, the compound (VIII) is represented by the formula (VIIIB)

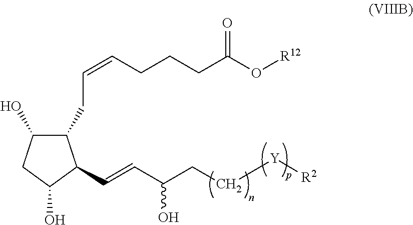

wherein R², Y, n and p have the same meaning as defined for the compound (I), and R¹² is C₁₋₆-alkyl.

In one embodiment of the invention, carboxyl protecting group of the obtained compound (VIII) is hydrolyzed upon strong base treatment, preferably lithium hydride, in the mixture of solvents such as methanol, ethanol, tetrahydrofurane, dioxane and water.

In one embodiment of the invention, fluprostenol is obtained, it is represented by the formula (IA), wherein X represents —O—; R¹ is H; n is 2, Y represents

—O—, p is 1, and R² is phenyl substituted in meta position by trifluoromethyl.

Optionally, acid (IA) obtained upon basic hydrolyzis is alkylated with C₁₋₃-alkyl halide in the presence of a strong base, such as 1,8-diazabicyclo[5.4.0]undec-7-en (DBU) or 1,5-diazabicyclo[4.3.0]non-5-en (DBN), to yield prostaglandin ester of the formula (IB)

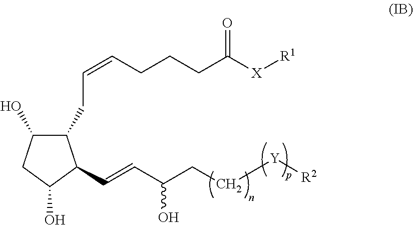

wherein
X represents —O—;
R¹ is C₁₋₃-alkyl;
R², Y, n and p have the same meaning as defined for the compound (I).

Following the protocol of the present invention, travoprost is obtained, it is represented by the formula (IB), wherein X represents —O—; R¹ is C₃-alkyl; n is 2, Y represents —O—, p is 1, a R² is phenyl substituted in meta position by trifluoromethyl.

The ester of the formula (IB) is reacting with R¹NH₂ amine, wherein R¹ is C₁₋₃-alkyl, yielding prostamid of the formula (IC)

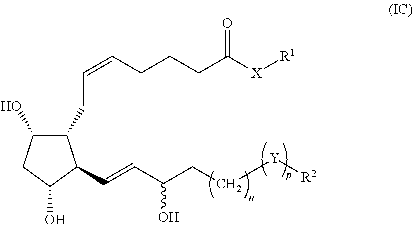

wherein
R¹ is C₁₋₃-alkyl;
R², Y, n and p have the same meaning as defined for the compound (I).

In this manner, using aqueous or alcoholic solution of ethylamine in amidation reaction, bimatoprost represented by the formula (I) is obtained, wherein

X represents —NH—; R¹ is C₁₋₃-alkyl; n is 1, p is 0, and R² is phenyl.

Preferably, direct amidation using the compound of the formula (VIII), obtained in step (e) is performed, which significantly shortens synthetic rout.

The present invention provides wide range of pharmacologically active F_{2α} prostaglandin analogues, using the same, chemically stable and structurally advanced synthon. Under the protocol of the present invention, laborious purification of the intermediates is avoided, which contributes to the costs decrease of the synthesis and enables the implementation of this process in a large plant scale. The main advantage of the present invention, in comparison with the standard methods, is obtaining expected ester of the formula (VIII) in high diastereoisomeric excess. In addition, the inseparable diastereoisomeric impurity accompanying the end product is detected only in trace amounts.

The small amounts of (15S)-(+) travoprost epimer can be removed by preparative HPLC and (15R)-(+) bimatoprost isomeric impurity can be easily discarded by crystallization, yielding the final products of pharmaceutical purity.

The invention is illustrated by the following examples.

### EXAMPLES

1-[(Z)-6-[(1R,2R,3R,5S)-2-[(Phenylsulfonyl)methyl]-3,5-bis(triethylsilyloxy)cyclopentyl]-4-hexenyl]-4-methyl-2,6,7-trioxabicyclo[2.2.2]acetate as the mixture of 5Z/5E isomers at the 91.55%:8.45%, 83.1% de ratio was used as the synthon.

### Analytical Methods

Thin layer chromatography (TLC). Thin layer chromatography was performed on aluminum silica gel covered plates (Kieselgel 60 F₂₅₄, Merck). The chromatography plates were visualized due to spraying with the solution of Ce(SO₄)₂ (10 g) and H₃[P(Mo₃O₁₀)₄] (20 g) in 10% H₂SO₄ aqueous solution (1 dm³) and then heating at 120° C.

Column chromatography. The compounds were purified by column chromatography, silica gel was used as the column filler (Kieselgel 60, 40-63 μm, 230-400 mesh, Merck). The mixture of ethyl acetate, methanol, 2-propanol and methylene dichloride at different ratios was used as the eluent.

HPLC. HPLC analyzes were performed on Waters 2695 liquid chromatograph, equipped with PDA Waters 2998 detector, on Gemini C18, AS-3R and Poroshell 120EC-C8 columns, using the mixture of acetonitrile, methanol and water at different rations as mobile phase.

HPLC-MS. HPLC-MS (ESI) analyzes were performed on Shimadzu LC-2010A HT liquid chromatograph, coupled with Applied Biosystems Qtrap 3200 mass spectrometer, on Gemini C18, AS-3R and Poroshell 120EC-C8 columns, using the mixture of acetonitrile, methanol and water at different rations as mobile phase.

Spectroscopic methods. ¹H NMR and ¹³C NMR spectra of the obtained compounds were recorded on NMR spectrometer type Varian VNMRS-600 (600 MHz), in C₆D₆ or CDCl₃ using TMS as the internal standard. Infrared spectra were recorded on Nicolet Imapct 410 FT-IR spectrophotometer.

High-Performance Mass Spectroscopy (HRMS). High performance mass spectra (EI, ESI) were recorded on AMD 604 by AMD Intectra Gmbh and Mariner by PE Biosystems equipped with time of flight analyzer (TOF) spectrophotometers.

Melting Point. Melting point was determined on the basis of DSC measurements recorded on differential scanning calorimeter DSC822E by Mettler Toledo.

Optical Rotation. Optical rotations were measured on automatic polarimeter Perkin Elmer 341. Measurements were carried out in ethanol, chloroform or CH₂Cl₂, concentrations are given in [%].

### Example 1

### Synthesis of (15R)-(+)-1a travoprost and its (1S)-(+)-(1b) epimer

### (R)-(−)-2,2-Dimethyl-4-(toluenesulfonyloxymethyl)-1,3-dioxolane (20a)

p-Toluenesulfonyl chloride (6.18 g, 31.748 mmol) was added portionwise over a period of 10 min to a solution of (S)-(+)-2,2-dimethyl-4-(hydroxymethyl)-1,3-dioxolane (19a) (4.00 g, 30.236 mmol) in anhydrous pyridine (50.0 ml) in an ice bath. The resulting solution was slowly brought to room temperature and stirred overnight. During that time, a white precipitate formed. The pyridine was removed under reduced pressure and the residue was diluted with ethyl acetate (50 ml), washed subsequently with cold aqueous 1M HCl (2×150 ml), saturated NaHCO₃ (100 ml) and brine (200 ml). The organic layer was dried over Na₂SO₄, filtered and concentrated to give a light yellow oil. The crude product was purified by column chromatography over silica gel with gradient elution 10-30% ethyl acetate/hexanes to afford (R)-(−)-3-tosyloxy-1,2-propanediol acetonide 20a (8.54 g, 98.6% yield, 99.88% ee) as a colourless viscosous oil. The liquid changes to a white solid at low temperatures. [α]_{D}²⁰=−4.75° (c 1.0, EtOH) (lit. [α]_{D}²⁴=−4.6° (c 13.0, EtOH))¹². FT-IR (thin film) νₘₐₓ (cm⁻¹): 3073, 2987, 2937, 2891, 1598, 1495, 1455, 1368, 1257, 1213, 1190, 1177, 1096, 1055, 979, 829, 788, 665, 555. ¹H NMR (600 MHz, CDCl₃, 25° C.) δ (ppm): 1.31 (s, 3H, CH₃-2), 1.34 (s, 3H, CH₃-2), 2.45 (s, 3H, ArCH₃), 3.76 (dd, J=5.1 and 8.8 Hz, 1H, one of the CH₂-5 group), 3.98 (dd, J=6.0 and 10.2 Hz, 1H, one of the CH₂-1′ group), 4.01 (dd, J=5.6 and 10.3 Hz, 1H, one of the CH₂-1′ group), 4.03 (dd, J=6.2 and 8.8 Hz, 1H, one of the CH₂-5 group), 4.28 (m, 1H, CH-4), 7.35 (m, 2H, aromatic H-3 and H-5), 7.79 (m, 2H, aromatic H-2 and H-6). ¹³C NMR (150 MHz, CDCl₃, 25° C.) δ (ppm): 21.54 (Ar—CH₃), 25.05 (CH₃-2), 26.53 (CH₃-2), 66.05 (C-5), 69.44 (C-1′), 72.82 (C-4), 109.93 (C-2), 127.88 (2C, aromatic C-2 and C-6), 129.83 (2C, aromatic C-3 and C-5), 132.55 (aromatic C-1), 144.99 (aromatic C-4). HRMS (ESI): calcd. for C₁₃H₁₈O₅NaS [M+Na]⁺ 309.07672; fund 309.0762.

Chiral HPLC: Chiralpak IA, 5 μm, 250×4.6 mm column, hexanes/2-propanol/methanol 97:2:1 (v/v/v), 1.0 ml/min, Rₜ=21.71 min. (0.06% yield of 20b), Rₜ=24.20 (99.94% yield of 20a), 99.88% ee.

### (S)-(+)-2,2-Dimethyl-4-(toluenesulfonyloxymethyl)-1,3-dioxolane (19b)

According to the procedure described for the preparation of (R)-(−)-20a, (R)-(−)-2,2-dimethyl-4-(hydroxymethyl)-1,3-dioxolane (19b) (2.50 g, 18.652 mmol) afforded the right-hand (S)-(+)-3-tosyloxy-1,2-propanediol acetonide 20b (5.23 g, 98.0% yield, 99.24% ee). [α]_{D}²⁰=+4.5° (c 1.0, EtOH) (lit. [α]_{D}²⁵=+4.7° (c 1.0, EtOH))¹³. The characterization data from IR, NMR and HRMS spectra were identical in all aspects with those of (R)-(−)-20a enantiomer.

### (S)-(+)-2,2-Dimethyl-4-(3-trifluoromethylphenoxy)methyl-1,3-dioxolane (21a)

### Method A.

Sodium hydroxide (1.73 g, 43.218 mmol) was added portionwise to a stirred solution of 3-trifluoromethylphenol (7.08 g, 43.218 mmol) in a mixture of EtOH and H₂O (4:1, 125 ml). After being stirred for 10 min, a solution of tosylate (R)-(−)-20a (8.25 g, 28.812 mmol) in EtOH (25 ml) was added dropwise and the reaction mixture was heated at reflux for 20 h to disappearance of the starting tosylate (R)-(−)-20a (TLC, hexanes/ethyl acetate 4:1). EtOH was then evaporated, the residue was treated with 10%0/NaOH (25 ml) and extracted with CH₂Cl₂ (3×25 ml). The combined organic layers were washed with H₂O (3×100 ml), dried over Na₂SO₄, filtered and evaporated to give a light yellow oil (7.80 g, 98% yield). The crude product was distilled to afforded the acetonide (S+)(+)-21a (7.06 g, 88.7% yield, 99.88% ee) as a colourless oil. bp 80-94° C. (0.2 mmHg). [α]_{D}²⁰=+7.53° (c 0.5, EtOH) (lit. [α]_{D}²⁵=+11° (c 0.5, EtOH))¹³. FT-IR (thin film) νₘₐₓ (cm⁻¹): 3074, 2988, 2938, 2883, 1608, 1593, 1493, 1450, 1372, 1330, 1233, 1168, 1127, 1096, 1066, 976, 904, 843, 793, 698, 657, 520. ¹H NMR (600 MHz, CDCl₃, 25° C.) δ (ppm): 1.41 (s, 3H, CH₃-2), 1.46 (s, 3H, CH₃-2), 3.91 (dd, J=5.8 and 8.5 Hz, 1H, one of the CH₂-5 group), 3.98 (dd, J=5.8 and 9.5 Hz, 1H, one of the CH₂-1′ group), 4.08 (dd, J=5.5 and 9.5, 1H, one of the CH₂-1′ group), 4.18 (dd, J=6.4 and 8.5 Hz, 1H, one of the CH₂-5 group), 4.49 (p, J=5.9 Hz, 1H, CH-4), 7.09 (dd, J=2.4 and 8.2 Hz, 1H, aromatic H-6), 7.15 (m, 1H, aromatic H-2), 7.22 (dm, J=7.6 Hz, 1H, aromatic H-4), 7.38 (dd, J=8.1 and 8.1 Hz, 1H, aromatic H-5). ¹³C NMR (150 MHz, CDCl₃, 25.3° C.) δ (ppm): 25.27 (CH₃-2), 26.72 (CH₃-2), 66.64 (C-5), 69.01 (C-1′), 73.84 (C-4), 109.89 (C-2), 111.38 (d, J=3.7 Hz, aromatic C-2), 117.80 (d, J=3.4 Hz, aromatic C-4), 118.0 (d, J=1.2 Hz, aromatic C-6), 123.88 (q, J=272.1 Hz, —CF₃), 129.97 (aromatic C-5), 131.86 (q, J=32.4 Hz, aromatic C-3), 158.66 (aromatic C-1). HRMS (EI-HR): calcd. for C₁₃H₁₅O₃F₃ 276.09733; fund 276.09712.

Chiral HPLC: Chiracel OJ, 10 μm, 250×4.6 mm column, hexanes/ethanol 100:0.5 (v/v), 1.0 ml/min, Rₜ=8.27 min. (0.06% yield of 21b), Rₜ=14.24 (99.94% yield of 21a), 99.88% ee.

### Method B.

A solution of (S)-(+)-2,2-dimethyl-4-(hydroxymethyl)-1,3-dioxolane (19a) (3.56 g, 26.952 mmol) and DIAD (6.98 ml, 33.69 mmol) in toluene (10 ml) was slowly added to a mixture of 3-trifluoromethylphenol (2.75 ml, 22.46 mmol) and PPh₃ (8.925 g, 33.69 mmol) in toluene (50 ml) at 90° C. over 30 min. After heating at 100° C. for another 1 h, TLC analysis (CH₂Cl₂/MeOH 20:1) indicated disappearance of the starting solketal 19a. The excess of toluene (30 ml) was evaporated and the residue was put into refrigerator for several hours. Triphenylphosphine oxide was removed by filtration on a Büchner funnel and washed with cold toluene (3×15 ml). The filtrate and washings were combined and concentrated under reduced pressure to give the crude product 21a (9.6 g) as an orange-yellow oil. The crude product was distilled to afforded the acetonide (S)-(+)-21a (5.98 g, 96.46% yield, 89.50% ee) as a colourless oil. bp 80-94° C. (0.2 mmHg). The characterization data from IR and NMR spectra were identical in all aspects with those of (S)-(+)-21a obtained according to the Method A.

### (R)-(−)-2,2-Dimethyl-4-(3-trifluoromethylphenoxy)methyl-1,3-dioxolane (21b)

In the same manner as described for the preparation of (S)-(+)-21a (Method A), the tosylate (S)-(+)-20b (5.0 g, 17.462 mmol) afforded the acetonide (R)-(−)-21b (4.13 g, 85.6% yield, 99.24% ee). [α]_{D}²⁰=−7.39° (c 0.5, EtOH). The characterization data from IR, NMR and HRMS spectra were identical in all aspects with those of (S)-(+)-21a enantiomer.

### (R)-(−)-3-(3-trifluoromethylphenoxy)propane-1,2-diol (22a)

1.0 M HCl (40.0 ml) was added in one portion to a solution of acetonide (S)-(+)-21a (6.90 g, 24.977 mmol) in acetone (50 ml). After heating at 70° C. for 1 h, TLC analysis (CH₂Cl₂/MeOH 20:1) indicated the reaction was complete. The solution was cooled, acetone was then evaporated and the aqueous acidic residue was slowly neutralized with slightly more than the equivalent amount of solid NaHCO₃. The resulting solution was extracted with CH₂Cl₂ (3×25 ml). The combined extracts were washed with water (3×150 ml), dried over Na₂SO₄, filtered and concentrated to give a light yellow oil (5.88 g). Purification by silica gel flash chromatography with CH₂Cl₂/MeOH (20:1) elution afforded the diol (R)-(−)-22a (5.79 g, 98.1% yield, 99.94% ee) as a white solid. mp 71.32-77.54° C., peak 74.21° C., heating rate 10.00° C./min (lit. mp 68-69° C.)¹³. [α]_{D}²⁰=−12.64° (c 1.0, EtOH). FT-IR (KBr) νₘₐₓ (cm⁻¹): 3318, 3224, 2954, 2927, 1607, 1495, 1451, 1341, 1244, 1178, 1123, 1054, 996, 902, 862, 793, 698, 659. ¹H NMR (600 MHz, CDCl₃, 25° C.) δ (ppm): 2.90 (br. s, 2H, two —OH groups), 3.74 (dd, J=5.7 and 11.4 Hz, 1H, one of the CH₂-1 group), 3.84 (dd, J=3.6 and 11.4 Hz, 1H, one of the CH₂-1 group), 4.04 (m, 2H, CH₂-3), 4.13 (m, 1H, CH-2), 7.07 (dd, J=2.6 and 8.3 Hz, 1H, aromatic H-6), 7.14 (m, 1H, aromatic H-2), 7.22 (dm, J=7.6 Hz, 1H, aromatic H-4), 7.36 (dd, J=8.0 and 8.0 Hz, 1H, aromatic H-5). ¹³C NMR (150 MHz, CDCl₃, 25° C.) δ (ppm): 63.50 (C-1), 69.27 (C-3), 70.35 (C-2), 111.35 (d, J=3.9 Hz, aromatic C-2), 117.93 (d, J=1.0 Hz, aromatic C-6), 117.97 (d, J=3.8 Hz, aromatic C-4), 123.82 (q, J=272.1 Hz, —CF₃), 130.08 (aromatic C-5), 131.92 (q, J=32.0 Hz, aromatic C-3), 158.50 (aromatic C-1). HRMS (EI-HR): calcd. for C₁₀H₁₁O₃F₃ 236.06603; fund 236.06637.

Chiral HPLC: Chiracel OD OD00CE-EL068, 10 μm, 250×4.6 mm column, hexanes/2-propanol 96:4 (v/v), 1.0 ml/min, Rₜ=25.21 min. (99.97% yield of 22a), Rₜ=31.14 (0.03% yield of 22b), 99.94% ee.

### (S)-(+)-3-(3-Trifluoromethylphenoxy)propane-1,2-diol (22b)

Treatment of the acetonide (R)-(−)-21b (4.0 g, 14.480 mmol) similar to the hydrolysis of (S)-(+)-21a afforded the diol (S)-(+)-22b (3.32 g, 97.1% yield, 99.23% ee). [α]_{D}²⁰=+12.45° (c 1.0, EtOH). The characterization data from IR, NMR and HRMS spectra were identical in all aspects with those of (R)-(−)-22a enantiomer.

### (S)-(+)-2-Hydroxy-3-(3-trifluoromethylphenoxy)propyl pivalate (23a)

Trimethylacetyl chloride (3.04 ml, 24.451 mmol) was added to a stirred solution of diol (R)-(−)-22a (5.50 g, 23.286 mmol) in a mixture of CH₂Cl₂ and pyridine (1:1, 50 ml) at 0° C. under an argon atmosphere. After stirring at 0° C. for 1 h and at room temperature for 1 h, the reaction was quenched with crushed ice (25 g) and the whole was portioned between AcOEt (50 ml) and 10% aqueous HCl (50 ml). The resulting layers were separated and the aqueous phase was extracted with AcOEt (3×25 ml). The combined organic extracts were washed successively with H₂O (150 ml), saturated aqueous NaHCO₃ (150 ml), brine (200 ml) and dried over anhydrous Na₂SO₄. Filtration and evaporation in vacuo furnished the crude ester (8.45 g), which was purified by flash column chromatography over silica gel (hexanes/AcOEt 4:1) to afford the pivalate (S)-(+)-23a (7.09 g, 95.1% yield, 99.86% ee) as a colourless oil. [α]_{D}²⁰=+1.90° (c 1.0, EtOH). FT-IR (thin film) νₘₐₓ (cm⁻¹): 3467, 3075, 2975, 2938, 2876, 1731, 1593, 1493, 1481, 1451, 1330, 1285, 1240, 1167, 1128, 1066, 1047, 999, 940, 884, 793, 698, 659. ¹H NMR (600 MHz, CDCl₃, 25° C.) δ (ppm): 1.21 (s, 9H, —C(CH₃)₃), 4.05 (dd, J=5.6 and 9.4 Hz, 1H, one of the CH₂-3 group), 4.07 (dd, J=4.5 and 9.5 Hz, 1H, one of the CH₂-3 group), 4.24 (m, 1H, CH-2), 4.28 (m, 2H, CH₂-1), 7.07 (dd, J=2.4 and 8.3 Hz, 1H, aromatic H-6), 7.14 (m, 1H, aromatic H-2), 7.23 (dm, J=7.9 Hz, 1H, aromatic H-4), 7.36 (dd, J=8.0 and 8.0 Hz, 1H, aromatic H-5). ¹³C NMR (150 MHz, CDCl₃, 25° C.) δ (ppm): 27.14 (3C, (CH₃)₃C—)), 38.88 ((CH₃)₃C—), 65.15 (C−1), 68.64 (C-2), 68.96 (C-3), 111.41 (d, J=3.8 Hz, aromatic C-2), 117.96 (d, J=1.0 Hz, aromatic C-6), 118.03 (d, J=3.8 Hz, aromatic C-4), 123.81 (q, J=271.6 Hz, —CF₃), 130.10 (aromatic C-5), 131.97 (q, J=32.3 Hz, aromatic C-3), 158.46 (aromatic C-1), 178.80 (C═O). HRMS (ESI): calcd. for C₁₅H₁₉O₄F₃Na [M+Na]⁺ 343.11277; fund 343.1134.

Chiral HPLC: Chiracel OD-H, 5 pun, 250×4.6 mm column, hexanes/ethanol/methanol 98:1.5:1 (v/v/v), 1.0 ml/min, Rₜ=11.09 min. (99.93% yield of 23a), Rₜ=12.60 (0.07% yield of 23b), 99.86% ee.

### (R)-(−)-2-Hydroxy-3-(3-trifluoromethylphenoxy)propyl pivalate (23b)

According to the procedure described for the preparation of (S)-(+)-23a, the diol (S)-(+)-22b (3.0 g, 12.702 mmol) yielded the pivalate (R)-(−)-23b (3.83 g, 94.2% yield, 99.20% ee). [α]_{D}²⁰=−1.45° (c 1.0, EtOH). The characterization data from IR, NMR and HRMS spectra were identical in all aspects with those of (S)-(+)-23a enantiomer.

### (S)-(−)-2-(tert-Butyldimethylsilyloxy)-3-(3-trifluoromethylphenoxy)propyl pivalate (24a)

tert-Butyldimethylsilyl chloride (4.02 g, 25.850 mmol) was added in one portion to a stirred solution of alcohol (S)-(+)-23a (6.90 g, 21.542 mmol) and imidazole (3.70 g, 53.855 mmol) in anhydrous DMF (50 ml) at 0° C. under an argon atmosphere. The reaction was allowed to proceed for 18 h at room temperature and then quenched with crushed ice (25 g). The resulting mixture was portioned between hexanes (50 ml) and H₂O (100 ml). The aqueous layer was extracted with hexanes (3×25 ml). The combined organic extracts were washed successively with H₂O (100 ml), brine (150 ml) and dried over Na₂SO₄. Filtration and evaporation in vacuo furnished the crude product (9.61 g) as a light yellow oil, which was purified by flash column chromatography (silica gel, 6%-10% hexanes/AcOEt) to give TBDMS ether (S)-(−)-24a (8.66 g, 92.5% yield, 99.88% ee) as a colourless oil. [α]_{D}²⁰=−6.66° (c 1.0, EtOH). FT-IR (thin film) νₘₐₓ (cm⁻¹): 3077, 2957, 2932, 2885, 2858, 1732, 1609, 1593, 1449, 1399, 1330, 1283, 1252, 1167, 1131, 1066, 1051, 1003, 880, 837, 780, 698. ¹H NMR (600 MHz, CDCl₃, 600 MHz, 25° C.) δ (ppm): 0.12 (s, 3H, CH₃—Si), 0.14 (s, 3H, CH₃—Si), 0.89 (s, 9H, (CH₃)₃C—Si), 1.21 (s, 9H, (CH₃)₃C—C), 3.96 (dd, J=6.0 and 9.5 Hz, 1H, one of the CH₂-3 group), 4.02 (dd, J=4.5 and 9.5 Hz, 1H, one of the CH₂-3 group), 4.12 (dd, J=6.7 and 13.1 Hz, 1H, one of the CH₂-1 group), 4.23 (m, 1H, one of the CH₂-1 group), 4.24 (m, 1H, CH-2), 7.07 (dd, J=2.2 and 8.4 Hz, 1H, aromatic H-6), 7.12 (m, 1H, aromatic H-2), 7.21 (dm, J=7.9 Hz, 1H, aromatic H-4), 7.39 (dd, J=7.9 and 7.9 Hz, 1H, aromatic H-5). ¹³C NMR (150 MHz, CDCl₃, 25° C.) δ (ppm): −4.85 (CH₃—Si), −4.65 (CH₃—Si), 18.03 ((CH₃)₃C—Si), 25.67 (3C, (CH₃)₃C—Si), 27.20 (3C, (CH₃)₃C—C), 38.81 ((CH₃)₃C—C), 65.40 (C−1), 69.06 (C-2), 69.82 (C-3), 111.19 (d, J=4.1 Hz, aromatic C-2), 117.65 (d, J=4.1 Hz, aromatic C-4), 118.04 (d, J=1.2 Hz, aromatic C-6), 123.91 (q, J=272.2 Hz, —CF₃), 130.01 (aromatic C-5), 131.91 (q, J=32.4 Hz, aromatic C-3), 158.80 (aromatic C-1), 178.26 (C═O). HRMS (ESI): calcd. for C₂₁H₃₃O₄F₃NaSi [M+Na]⁺ 457.19924; fund 457.1973.

Chiral HPLC: Chiracel OD-H, 5 μm, 250×4.6 mm column, hexanes/2-propanol 100:1 (v/v), 1.0 ml/min, Rₜ=7.08 min. (0.06% yield of 24b), Rₜ=7.49 (99.94% yield of 24a), 99.88% ee.

### (R)-(+)-2-(tert-Butyldimethyilyloxy)-3-(3-trifluoromethylphenoxy)propyl pivalate (24b)

In the same manner as described for the preparation of (S)-(−)-24a, the alcohol (R)-(−)-23b (3.60 g, 11.239 mmol) afforded the ether (R)-(+)-24b (4.46 g, 91.4% yield, 99.21% ee). [α]_{D}²⁰=+6.39° (c 1.0, EtOH). The characterization data from IR, NMR and HRMS spectra were identical in all aspects with those of (S)-(−)-24a enantiomer.

### (R)-(−)-2-(tert-Butyldimethylilyloxy)-3-(3-trifluoromethylphenoxy)propan-1-ol (25a)

Diisobutylaluminum hydride in toluene (1.0 M, 49.0 ml, 49.00 mmol) was added dropwise over 20 min. to a stirred solution of pivalate (S)-(−)-24a (8.45 g, 19.445 mmol) in anhydrous CH₂Cl₂ (100 ml) at −78° C. under an argon atmosphere. The resulting mixture was allowed to warm to −20° C. for a 30 min period and stirred at this temperature for another 2 h. TLC analysis (hexanes/AcOEt 8:1) indicated disappearance of the starting pivalate (S)-(−)-24a. The clear colourless solution was re-cooled to −78° C. and the excess of DIBAL was quenched by addition of MeOH (25 ml) dropwise. On warming to 0° C., 10% aqueous potassium sodium tartrate (150 ml) was added and the mixture was stirred vigorously at room temperature for 2 h. The resulting layers were separated and the aqueous phase was extracted with CH₂Cl₂ (3×75 ml). The combined extracts were washed with water (150 ml), brine (200 ml) and dried over anhydrous Na₂SO₄. Filtration and evaporation in vacuo furnished the crude product (7.29 g), which was purified by flash column chromatography (silica gel, 3%-11% hexanes/AcOEt) to afford the primary alcohol (R)-(−)-25a (6.28 g, 92.2% yield, 99.90% ee) as a colourless oil. [α]_{D}²⁰=−29.36° (c 1.0, EtOH). FTIR (thin film) νₘₐₓ (cm⁻¹): 3434, 3074, 2954, 2931, 2886, 2858, 1608, 1592, 1493, 1449, 1330, 1254, 1169, 1130, 1066, 1048, 999, 881, 837, 781, 697, 659. ¹H NMR (600 MHz, CDCl₃, 25° C.) δ (ppm): 0.13 (s, 3H, CH₃—Si), 0.15 (s, 3H, CH₃—Si), 0.92 (s, 9H, (CH₃)₃C—Si), 2.02 (br. s, 1H, —OH), 3.69 (dd, J=4.2 and 11.2 Hz, 1H, one of the CH₂-1 group), 3.74 (dd, J=4.1 and 11.4 Hz, 1H, one of the CH₂-1 group), 3.96 (dd, J=6.3 and 9.3, 1H), 4.04 (dd, J=5.4 and 9.3 Hz, 1H, one of the CH₂-3 group), 4.13 (m, 1H, CH-2), 7.07 (dd, J=2.6 and 8.2 Hz, 1H, aromatic H-6), 7.12 (m, 1H, aromatic H-2), 7.21 (dm, J=7.7, 1H, aromatic H-4), 7.38 (dd, J=8.0 and 8.0 Hz, 1H, aromatic H-5). ¹³C NMR (150 MHz, CDCl₃, 25° C.) δ (ppm): −4.86 (CH₃—Si), −4.55 (CH₃—Si), 18.09 ((CH₃)₃C—Si), 25.77 (3C, (CH₃)₃C—Si), 64.09 (C−1), 69.27 (C-3), 71.11 (C-2), 111.1 (d, J=3.7 Hz, aromatic C-2), 117.6 (d, J=4.0 Hz, aromatic C-4), 118.0 (d, J=1.2 Hz, aromatic C-6), 123.91 (q, J=272.1 Hz, —CF₃), 129.98 (aromatic C-5), 131.88 (q, J=32.3 Hz, aromatic C-3), 158.76 (aromatic C-1). HRMS (ESI): calcd. for C₁₆H₂₅O₃F₃NaSi [M+Na]⁺ 373.14173; fund 373.1420.

Chiral HPLC: Chiracel OD-H, 5 μm, 250×4.6 mm column, hexanes/2-propanol 100:0.5 (v/v), 1.0 ml/min, Rₜ=15.96 min. (0.05% yield of 25b), Rₜ=21.88 (99.95% yield of 25a), 99.90% ee.

### (S)-(+2-(tert-Butyldimethysilyloxy)-3-(3-trifluoromethylphenoxy)propan-1-ol (25b)

Treatment of the pivalate (R)-(+)-24b (4.2 g, 9.665 mmol) similar to the reduction of (S)-(−)-24a afforded the primary alcohol (S)-(+)-25b (3.07 g, 90.7% yield, 99.18% ee). [α]_{D}²⁰=+28.48° (c 1.0, EtOH). The characterization data from IR, NMR and HRMS spectra were identical in all aspects with those of (R)-(−)-25a enantiomer.

### (S)-(−)-2-(tert-Butyldimethylsilyloxy)-3-(3-trifluoromethylphenoxy)propanal (16a)

Dess-Martin periodinane (9.06 g, 20.717 mmol) was added portionwise to a cold (0° C.) suspension of alcohol (R)-(−)-25a (6.05 g, 17.264 mmol) and dry NaHCO₃ (4.35 g, 51.792 mmol) in anhydrous CH₂Cl₂ (100 ml). After being stirred for 1 h at room temperature, TLC analysis (hexanes/AcOEt 9:1) indicated disappearance of the starting alcohol (R)-(−)-25a. Saturated aqueous NaHCO₃ (100 ml) and Na₂SO₃ (15.23 g, 120.848 mmol) were then added simultaneously and the mixture was stirred at room temperature for 30 min. The resulting layers were separated and the aqueous phase was extracted with CH₂Cl₂ (3×50 ml). The combined extracts were washed with water (3×150 ml), dried over Na₂SO₄, filtered and concentrated in vacuo. The crude product was co-evaporated with anhydrous tetrahydrofuran (3×50 ml) and carefully dried under reduced pressure to afford the crude aldehyde (S)-(−)-16a (5.82 g, 96.7%) as a light yellow oil. The aldehyde (S)-(−)-16a was directly used for the next step without further purification. [α]_{D}²⁰=−26.41° (c 1.0, CHCl₃). FT-IR (thin film) νₘₐₓ (cm⁻¹): 3077, 2954, 2932, 2886, 2859, 1740, 1593, 1493, 1450, 1330, 1254, 1169, 1130, 976, 881, 838, 782, 697, 658. ¹H NMR (CDCl₃, 600 MHz) δ (ppm): 0.13 (s, 3H, CH₃—Si), 0.17 (s, 3H, CH₃—Si), 0.94 (s, 9H, (CH₃)₃C—Si), 4.13 (dd, J=6.6 and 9.9 Hz, 1H, one of the CH₂-3 group), 4.25 (dd, J=3.6 and 9.9 Hz, 1H, one of the CH₂-3 group), 4.40 (ddd, J=0.8, 3.6 and 6.6 Hz, 1H, CH-2), 7.07 (dd, J=2.4 and 8.3 Hz, 1H, aromatic H-6), 7.12 (m, 1H, aromatic H-2), 7.23 (dm, J=7.7 Hz, 1H, aromatic H-4), 7.39 (dd, J=8.1 and 8.1 Hz, 1H, aromatic H-5), 9.75 (d, J=0.8 Hz, 1H, —CHO). ¹³C NMR (150 MHz, CDCl₃, 25° C.) δ (ppm): −4.87 (CH₃—Si), −4.74 (CH₃—Si), 18.21 ((CH₃)₃C—Si), 25.66 (3C, (CH₃)₃C—Si), 69.20 (C-3), 76.70 (C-2), 111.30 (d, J=3.5 Hz, aromatic C-2), 117.68 (d, J=4.0 Hz, aromatic C-4), 118.00 (d, J=1.2 Hz, aromatic C-6), 123.90 (q, J=272.1 Hz, —CF₃), 130.05 (aromatic C-5), 132.00 (q, J=32.2 Hz, aromatic C-3), 158.52 (aromatic C-1), 202.01 (—CHO).

### (R)-(+)-2-(tert-Butyldimethylsilyloxy)-3-(3-trifluoromethylphenoxy)propanal (16b)

According to the procedure described for the preparation of (S)-(−)-16a, the alcohol (S)-(+)-25b (3.80 g, 10.843 mmol) yielded the crude aldehyde (R)-(+)-16b (3.60 g, 95.4% yield). [α]_{D}²⁰=+26.23° (c 1.0, CHCl₃). The characterization data from IR and NMR spectra were identical in all aspects with those of (S)-(−)-16a enantiomer.

### 1-[(4Z)-6-[(1R,2R,3R,5S)-2-[(1R/1S,2R/2S,3R)-3-(tert-Buthyldimethylsilyloxy)-4-[3-(trifluo methyl)phenoxy]-1-(phenylsulfonyl)butyl]-35-bis(triethylsilyloxy)cyclo-pentyl]-4-hexenyl]-4-methyl-2,6,7-trioxabicyclo[2.2.2]octan (3a)

To the solution of diisopropylamine (6.0 ml, 42.24 mmol) in anhydrous THF (12 ml) cooled to −60° C., stirred under argon atmosphere, n-BuLi solution (25.5 ml, 40.8 mmol, 1.6 M in hexane) was added dropwise, followed by addition of 1-[(Z)-6-[(IR,2R,3R,5S)-2-[(phenylsulfonyl)methyl]-3,5-bis(triethylsilyloxy)cyclopentyl]-4-hexenyl]-4-methyl-2,6,7-trioxabicyclo[2.2.2]octan LA-5 (8.8 g, 12.66 mmol, mixture of 5Z/5E isomers 91.55%:8.45%, 83.1% de) in anhydrous THF (15 ml). Resulting mixture was stirred at −60° C. for 30 min. The crude aldehyde (S)-(−)-2a (14.6 g, 41.90 mmol) in anhydrous THF (10 ml) was added. After 20 min. the cooling bath was removed, brine (20 ml) was added. Water and ether phases were separated. Water phase was extracted with THF (3×25 ml). Combined organic extracts were dried over anhydrous Na₂SO₄ (20 g). Drying agent was filtered off and the filtrate was condensed under vacuum. Obtained diastereoisomeric mixture of hydroxysulfones (15R)-3a (16.72 g) was used in the next steps without purification.

### 1-[(4Z)-6-[(1R,2R,3R,5S)-2-[(1R/1S,2R/2S,3S)-3-(tert-Buthyldimethylsilyloxy)-4-[3-(trifluoromethyl)phenoxy]-1-(phenylsulfonyl)butyl]-3,5-bis(triethylsilyloxy)cyclo-pentyl]-4-hexenyl]-4-methyl-2,6,7-trioxabicyclo[2.2.2]octan (3b)

Analogously, using sulfone LA-5 (7.1 g, 10.21 mmol, 83.1% de) and the crude aldehyde (S)-(+)-2b (12.0 g, 34.44 mmol), (15S)-3b hydroxysulfones (15.96 g) as the crude diastereomeric mixture were obtained.

### 1-[(4Z)-6-[(1R,2R,3R,5S)-2-[(3R)-3-(tert-Buthyldimethylsilyloxy)-4-[3-(trifluoromethyl)phenoxy]-1-butenyl]-3,5-bis(triethylsilyloxy)cyclopentyl]-4-hexenyl]-4-methyl-2,6,7-trioxabicyclo[2.2.2]octan (4a)

The saturated methanolic solution of Na₂HPO₄ (60 ml) was added to the crude mixture of hydroxysulfones (15R)-3a (16.72 g) in THF (20 ml), then sodium amalgam (21.0 g, 182.69 mmol Na, 20%) was added portionwise during 4 h. The resulting mixture was stirred at ambient temperature for 16 h. The solution was decantated and condensed under vacuum. After water (80 ml) and ethyl acetate (80 ml) addition two phases were formed. The product was extracted from water phase with ethyl acetate (3×50 ml). The combined organic extracts were dried over anhydrous Na₂SO₄ (30 g). Drying agent was filtered off and the filtrate was condensed under vacuum. The crude (15R)-4a olefin (14.44 g) was obtained and it was used in the next steps without purification.

### 1-[(4Z)-6-[(1R,2R,3R,5S)-2-[(3S)-3-(tert-Buthyldimethylsilyloxy)-4-[3-(trifluorome-thyl)phenoxy]-1-butenyl]-3,5-bis(triethylsilyloxy)cyclopentyl]-4-hexenyl]-4-methyl-2,6,7-trioxabicyclo[2.2.2]octan (4b)

Analogously, using the crude mixture of (15S)-3b hydrosulfones (15.96 g), crude (15S)-4b olefin (12.92 g) was obtained.

### 2,2-bis(Hydroxymethyl)propyl (5Z)-7-[(1R,2R,3R,5S)-3,5-dihydroxy-2-[(1E,3R)-3-hydroxy-4-[3-(trifluoromethyl)-phenoxy]-1-butenyl]cyclopentyl]-5-heptenate (5a)

To the solution of the crude silylated (15R)-4a prostaglandin derivative (14.44 g) in anhydrous THF (20 ml), tetrabutyloammonium fluoride (30 ml, 1.0 M in THF) was added dropwise. The resulting mixture was heated at 60° C. for 2 h. When the reaction was completed THF was removed, the oily residue was diluted with 10% citric acid aqueous solution (60 ml) to remove 4-methyl-OBO protecting group. After 15 min. the reaction product was salted out with sodium chloride, separated and dried under reduced pressure. The crude product (17.48 g) was purified by column chromatography (silica gel, methanol/AcOEt, in concentration gradient 2%-6%) to yield pentaol (15R)-(+)-5a (6.17 g, 87.0% yield of LA-S, 5a:5b:(5E,15R)-isomer=91.36%:0.18%:8.46%). [α]_{D}²⁰=+18.72° (c 1.0, CHCl₃). FT-IR (thin film) νₘₐₓ (cm⁻¹): 3369, 2934, 1716, 1592, 1493, 1451, 1330, 1240, 1167, 1125, 1040, 972, 792, 698. ¹H NMR (CDCl₃, 600 MHz, 25° C.) δ (ppm): 0.82 (s, 3H, —CH₃), 1.50 (m, 1H, CH-1 cyclopentyl ring), 1.66-1.70 (m, 3H, CH₂-3 of α chain and one proton of CH₂-4 group of cyclopentyl ring), 2.04 (m, 2H, one proton of CH₂-4 group of α chain and one proton of CH₂-7 group of α chain), 2.11 (m, 1H, one proton of CH₂-4 group of α chain), 2.24-2.34 (m, 5H, one proton of CH₂-7 and CH₂-2 group of α chain, one proton of CH₂-4 and CH-2 group of cyclopentyl ring), 3.51 (s, 4H, two —H₂OH groups), 3.92 (m, 1H, CH-3 of cyclopentyl ring), 3.97 (m, 2H, CH₂-4 of ω chain), 4.07 (s, 2H, CH₂-1 of α chain), 4.11 (m, 1H, CH-5 of cyclopentyl ring), 4.49 (m, 1H, CH-3 of ω chain), 5.32 (m, 1H, CH-5 of α chain), 5.40 (m, 1H, CH-6 of α chain), 5.66 (m, 2H, CH-1 and CH-2 of ω chain), 7.08 (dd, J=2.4 and 8.4 Hz, 1H, CH-6 aromatic), 7.14 (m, 1H, CH-aromatic), 7.20 (d, J=7.2 Hz, 1H, CH-4 aromatic), 7.37 (m, 1H, CH-5 aromatic). ¹³C NMR (150 MHz, CDCl₃, 25° C.) δ (ppm): 16.71 (—CH₃), 24.56 (C-3 of α chain), 25.47 (C-7 of α chain), 26.39 (C-4 of α chain), 33.33 (C-2 of α chain), 40.47 (—C(CH₃)(CH₂OH)₂), 42.80 (C-4 of cyclopentyl ring), 49.69 (C-1 of cyclopentyl ring), 55.44 (C-2 of cyclopentyl ring), 66.46 (2C, —C(CH₃)(CH₂OH)₂), 66.50 (CH₂-1 of α chain), 70.97 (C-3 of ω chain), 71.87 (C-4 of ω chain), 72.39 (C-5 of cyclopentyl ring), 77.35 (C-3 of cyclopentyl ring), 111.44 (q, J=4.0 Hz, C-2 aromatic), 117.67 (q, J=4.0 Hz, C-4 aromatic), 118.01 (C-6 aromatic), 124.37 (q, J=273.0 Hz, —CF₃), 129.20 (C-6 of α chain), 129.38 (C—S of α chain), 130.02 (C-5 aromatic), 130.26 (C-2 of ω chain), 131.77 (q, J=32.0 Hz, C-3 aromatic), 135.53 (C-1 of ω chain), 158.68 (C-1 aromatic), 174.59 (C═O). HRMS (ESI): calculated for C₂₈H₃₉O₅F₃Na [M+Na]⁺ 583.24892. found 583.2495.

HPLC: Gemini C18, 3 μm, 250×4.6 mm, KH₂PO₄ (4 g/l):CH₃CN:MeOH (90:5:5) (phase A)/CH₃CN (phase B), concentration gradient 67%-10%, 1.0 ml/min, Rₜ=36.13 min. (8.46%—(5E,15R)-isomer) Rₜ=37.96 min. (0.18%—(15S)-(+)-5b), Rₜ=39.62 min. (91.36%—(15R)-(+)-5a).

HPLC-MS (ESI): Gemini C18, 3 μm, 250×4.6 mm, KH₂PO₄ (4 g/l):CH₃CN:MeOH (90:5:5) (phase A)/CH₃CN (phase B), concentration gradient 67%-10%, 1.0 ml/min, Rₜ=37.33 min. (m/z=561.2 [M+H]⁺—(5E,15R)-isomer), Rₜ=37.90 min. (m/z=561.2 [M+H]⁺—(15S)-(+)-5b), Rₜ=40.56 min. (m/z=561.2 [M+H]⁺—(15R)-(+)-5a).

### 2,2-bis(Hydroxymethyl)propyl (5Z)-7-[(1R,2R,3R,5S)-3,5-dihydroxy-2-[(1E,3S)-3-hydroxy-4-[3-(trifluoromethyl)-phenoxy]-1-butenyl]cyclopentyl]-5-heptenate (5b)

Following the same procedure, using 12.92 g of crude (15S)-(+)-4b olefin, (15S)-(+)-5b pentaol (5E,15S)-isomer=0.53%:91.1%:8.37%) was obtained in 4.90 g (85.6%) yield, calculated on LA-5, 5a:5b. [α]_{D}²⁰=+23.80° (c 1.0, CHCl₃). FT-IR (thin film) νₘₐₓ (cm⁻¹): 3368, 2962, 2935, 2879, 1726, 1592, 1493, 1450, 1330, 1241, 1167, 1125, 1039, 972, 916, 882, 792, 699. ¹H NMR (CDCl₃, 600 MHz, 25° C.) δ (ppm): 0.84 (s, 3H, —CH₃), 1.55 (m, 1H, CH-1 of cyclopentyl ring), 1.70 (m, 2H, CH₂-3 of α chain), 1.80 (m, 1H, one proton of CH₂-4 group of cyclopentyl ring), 2.12-2.24 (m, 4H, CH₂-4 and one proton of CH₂-7 group of α chain, one proton of CH₂-4 group of cyclopentyl ring), 2.24-2.38 (m, 3H, CH₂-2 and one proton of CH₂-7 group of α chain), 2.42 (m, 1H, CH-2 of cyclopentyl ring), 3.53 (s, 4H, two —CH₂OH groups), 3.95 (dd, J=7.7 and 9.4 Hz, one proton of CH₂-4 group of ω chain), 3.97 (m, 1H, CH-3 of cyclopentyl ring), 4.04 (dd, J=3.8 and 9.4 Hz, one proton of CH₂-4 group of ω chain), 4.09 (d, J=11.5 Hz, one proton of CH₂-1 group of α chain), 4.11 (d, J=11.5 Hz, one proton of CH₂-1 group of α chain), 4.15 (m, 1H, CH-5 of cyclopentyl ring), 4.54 (m, 1H, CH-3 of ω chain), 5.35 (m, 1H, CH-5 of α chain), 5.45 (m, 1H, CH-6 of α chain), 5.70 (dd, J=5.6 i 15.5 Hz, CH-2 of ω chain), 5.72 (dd, J=8.3 and 15.5 Hz, CH-1 of ω chain), 7.10 (dd, J=2.1 i 8.4 Hz, 1H, CH-6 aromatic), 7.15 (m, 1H, CH-2 aromatic), 7.21 (d, J=8 Hz, 1H, CH-4 aromatic), 7.38 (m, 1H, CH-5 aromatic). ¹³C NMR (150 MHz, CDCl₃, 25° C.) δ (ppm): 16.82 (—CH₃), 24.64 (C-3 of α chain), 25.68 (C-7 of α chain), 26.44 (C-4 of α chain), 33.39 (C-2 of α chain), 40.56 (—C(CH₃)(CH₂OH)₂), 42.91 (C-4 of cyclopentyl ring), 50.31 (C-1 of cyclopentyl ring), 55.65 (C-2 of cyclopentyl ring), 66.60 (2C, —C(CH₃)(CH₂OH)₂), 66.68 (CH₂-1 of α chain), 70.50 (C-3 of ω chain), 72.08 (C-4 of ω chain), 72.89 (C-5 of cyclopentyl ring), 77.88 (C-3 of cyclopentyl ring), 111.51 (q, J=3.8 Hz, C-2 aromatic), 117.71 (q, J=3.8 Hz, C-4 aromatic), 118.08 (s, C-6 aromatic), 123.90 (q, J=271.0 Hz, —CF₃), 129.34 (C-6 of α chain), 129.42 (C-5 of α chain), 129.62 (C-2 of ω chain), 130.03 (C-5 aromatic), 131.82 (q, J=32.2 Hz, C-3 aromatic), 134.66 (C-1 of ω chain), 158.72 (C-1 aromatic), 174.56 (C═O). HRMS (ESI): calculated for C₂₈H₃₉O₈F₃Na [M+Na]⁺ 583.24892. found 583.2507.

HPLC: Gemini C18, 3 μm, 250×4.6 mm, KH₂PO₄ (4 g/l):CH₃CN:MeOH (90:5:5) (phase A)/CH₃CN (phase B) in concentration gradient 67%-10%, 1.0 ml/min, Rₜ=35.43 min. (8.37%—(5E,15S)-isomer, Rₜ=37.96 min. (91.1%—(15S)-(+)-5b), Rₜ=39.62 min. (0.53%—(15R)-(+)-5a).

HPLC-MS (ESI): Gemini C18, 3 μm, 250×4.6 mm, KH₂PO₄ (4 g/l):CH₃CN:MeOH (90:5:5) (phase A)/CH₃CN (phase B) in concentration gradient 67%-10%, 1.0 ml/min, Rₜ=35.68 min. (m/z=561.2 [M+H]⁺—(5E,15S)-isomer), Rₜ=37.90 min. (m/z=561.2 [M+H]⁺—(15S)-(+)-5b), Rₜ=40.50 min. (m/z=561.2 [M+H]⁺—(15R)-(+)-5a).

### (5Z)-7-[(1R,2R,3R,5S)-3,5-Dihydroxy-2-[(1E,3R)-3-hydroxy-4-[3-(trifluoro-methyl)phenoxy]-1-butenyl]cyclopentyl]-5-heptenoic acid (6a)

LiOH.H₂O (1.78 g, 42.455 mmol) was added to the solution of (15R)-(+)-5a ester (3.4 g, 6.065 mmol) in methanol (15 ml). The resulting slurry was stirred at ambient temperature for 24 h. Methanol was removed under reduced pressure, the residue was dissolved in water (150 ml) and acidified to pH 3-4 by addition of citric acid. The reaction product was extracted with ethyl acetate (4×25 ml). The combined organic extracts were dried over anhydrous Na₂SO₄ (10 g). The drying agent was filtered off and the filtrate was condensed under reduced pressure. The crude product (3.1 g) was purified by column chromatography (silica gel, methanol/AcOEt, 10%-80% concentration gradient), yielding fluprostenol (15R)-(+)-6a (2.72 g, 97.0%, 6a:6b:(5E,15R)-isomer=91.84%:0.18%:7.98%). [α]_{D}²⁰=+21.100 (c 1.0, CHCl₃). FT-IR (thin film) νₘₐₓ (cm⁻¹): 3363, 3009, 1710, 1593, 1493, 1451, 1330, 1239, 1168, 1125, 1066, 1035, 972, 913, 882, 792, 698. ¹H NMR (CDCl₃, 600 MHz, 25° C.) δ (ppm): 1.50 (m, 1H, CH-1 of cyclopentyl ring), 1.64 (m, 2H, CH₂-3 of α chain), 1.71 (m, 1H, one proton of CH₂-4 group of cyclopentyl ring), 2.09-2.11 (m, 3H, CH₂-4 and one proton of CH₂-7 group of α chain), 2.19-2.40 (m, 5H, CH₂-2 and one proton of CH₂-7 group of α chain, one proton of CH₂-4 group and CH-2 of cyclopentyl ring), 3.93 (m, 1H, CH-3 of cyclopentyl ring), 3.98 (m, 2H, CH₂-4 of ω chain), 4.14 (m, 1H, CH-5 of cyclopentyl ring), 4.52 (m, 1H, CH-3 of ω chain), 5.35 (m, 1H, CH-5 of α chain), 5.44 (m, 1H, CH-6 of α chain), 5.67 (m, 2H, CH-1 and CH-2 of ω chain), 7.09 (dd, J=2.1 and 8.3 Hz, aromatic CH-6), 7.15 (m, 1H, aromatic CH-2), 7.21 (d, J=7.7 Hz, aromatic CH-4), 7.38 (m, 1H, aromatic CH-5). ¹³C NMR (150 MHz, CDCl₃, 25° C.) δ (ppm): 24.47 (C-3 of α chain), 25.14 (C-7 of α chain), 26.27 (C-4 of α chain), 32.95 (C-2 of α chain), 42.59 (C-4 of cyclopentyl ring), 50.01 (C-1 of cyclopentyl ring), 55.32 (C-2 of cyclopentyl ring), 70.69 (C-3 of ω chain), 71.81 (C-4 of ω chain), 72.14 (C-5 of cyclopentyl ring), 77.20 (C-3 of cyclopentyl ring), 111.41 (q, J=3.8 Hz, aromatic C-2), 117.66 (q, J=4.0 Hz, aromatic C-4), 118.0 (aromatic C-6), 123.86 (q, J=271.2 Hz, —CF₃), 128.93 (C-6 of α chain), 129.60 (C-5 of α chain), 129.93 (C-2 of ω chain), 130.0 (aromatic C-5), 131.77 (q, J=32.2 Hz, aromatic C-3), 135.19 (C-1 of ω chain), 158.69 (aromatic C-1), 176.75 (C═O). HRMS (ESI): calculated for C₂₃H₂₉O₆F₃Na [M+Na]⁺ 481.18084. found 481.1802.

HPLC: Gemini C18, 3 μm, 250×4.6 mm, concentration gradient 67%-10% KH₂PO₄ (4 g/l):CH₃CN:MeOH (90:5:5) (phase A)/CH₃CN (phase B), 1.0 ml/min, Rₜ=30.43 min. (7.98%—(5E,15R)-isomer), Rₜ=33.18 min. (0.18%—(15S)-(+)-6b), Rₜ=34.23 min. (91.84%—(15R)-(+)-6a).

HPLC-MS (ESI): Gemini C18, 3 μm, 250×4.6 mm, concentration gradient 67%-10% KH₂PO₄ (4 g/l):CH₃CN:MeOH (90:5:5) (phase A)/CH₃CN (phase B), 1.0 ml/min, Rₜ=36.06 min. (m/z=497.4 [M+H]⁺—(5E,15R)-isomer), Rₜ=37.87 min. (m/z=497.4 [M+H]⁺—(15S)-(+)-6b), Rₜ=38.62 min. (m/z=497.4 [M+H]⁺—(15R)-(+)-6a).

### (5Z)-7-[(1R,2R,3R,5S)-3,5-Dihydroxy-2-[(E,3S)-3-hydroxy-4-[3-(trifluoro-methyl)phenoxy]-1-butenyl]cyclopentyl]-5-heptenoic acid (6b)

In the same manner, using (15S)-(+)-5b ester (3.10 g, 5.530 mmol), (15S)-(+)-6b acid was obtained in 2.44 g yield (96.4%, 6a:6b:(5E,15S)-isomer=0.53%:91.37%:8.10%). [α]_{D}²⁰=+23.95° (c 1.0, CHCl₃). FT-IR (thin film) νₘₐₓ (cm⁻¹): 3370, 3009, 2935, 1709, 1593, 1450, 1330, 1239, 1168, 1126, 1066, 1036, 972, 913, 882, 792, 698. ¹H NMR (CDCl₃, 600 MHz, 25° C.) δ (ppm): 1.48 (m, 1H, CH-1 of cyclopentyl ring), 1.63 (m, 2H, CH₂-3 of α chain), 1.77 (m, 1H, one proton of CH₂-4 group of cyclopentyl ring), 2.10 (m, 2H, CH₂-4 of α chain), 2.18 (m, 2H, CH₂-7 of α chain), 2.22-2.29 (m, 3H, one proton of CH₂-4 group of cyclopentyl ring and CH₂-2 of α chain), 2.38 (m, 1H, CH-2 of cyclopentyl ring), 3.95 (dd, J=7.4 i 9.4 Hz, one proton of CH₂-4 group of α chain), 3.98 (m, 1H, CH-3 of cyclopentyl ring), 4.02 (dd, J=3.5 i 9.4 Hz, one proton of CH₂-4 group of ω chain), 4.18 (m, 1H, CH-5 of cyclopentyl ring), 4.56 (m, 1H, CH-3 of ω chain), 5.35 (m, 1H, CH-5 of α chain), 5.47 (m, 1H, CH-6 of α chain), 5.68 (dd, J=5.6 i 15.4 Hz, CH-2 of ω chain), 5.71 (dd, J=8.2 and 15.4 Hz, CH-1 of ω chain), 7.07 (dd, J=2.1 i 8.4 Hz, 1H, aromatic CH-6), 7.13 (m, 1H, aromatic CH-2), 7.20 (d, J=8.0 Hz, 1H, aromatic CH-4), 7.36 (m, 1H, aromatic CH-5). ¹³C NMR (150 MHz, CDCl₃, 25° C.) δ (ppm): 24.44 (C-3 of α chain), 25.07 (C-7 of α chain), 26.20 (C-4 of α chain), 32.78 (C-2 of α chain), 42.90 (C-4 of cyclopentyl ring), 50.60 (C-1 of cyclopentyl ring), 55.56 (C-2 of cyclopentyl ring), 70.78 (C-3 of ω chain), 71.85 (C-4 of ω chain), 72.58 (C-5 of cyclopentyl ring), 77.65 (C-3 of cyclopentyl ring), 111.56 (q, J=4.1 Hz, aromatic C-2), 117.84 (q, J=3.6 Hz, aromatic C-4), 118.09 (aromatycic C-6), 123.88 (q, J=272.4 Hz, —CF₃), 128.97 (C-6 of α chain), 129.52 (C-2 of ω chain), 129.70 (C-5 of α chain), 130.08 (aromatic C-5), 131.85 (q, J=32.3 Hz, aromatic C-3), 135.07 (C-1 of ω chain), 158.63 (aromatic C-1), 177.29 (C═O). HRMS (ESI): calculated for C₂₃H₂₉O₆F₃Na [M+Na]⁺ 481.18084. found 481.1809.

HPLC: Gemini C18, 3 μm, 250×4.6 mm, KH₂PO₄ (4 g/l): CH₃CN:MeOH (90:5:5) (phase A)/CH₃CN (phase B), concentration gradient 67%-10%, 1.0 ml/min, Rₜ=29.52 min. (8.10%—(5E,15S)-isomer), Rₜ=33.18 min. (91.37%—(15S-(+)-6b), Rₜ=34.23 min. (0.53%—(15R)-(+)-6a).

HPLC-MS (ESI): Gemini C18, 3 μm, 250×4.6 mm, KH₂PO₄ (4 g/l):CH₃CN:MeOH (90:5:5) (phase A)/CH₃CN (phase B), concentration gradient 67%-10%, 1.0 ml/min, Rₜ=33.82 min. (m/z=497.4 [M+H]⁺—(5E,15S)-isomer), Rₜ=37.87 min. (m/z=497.4 [M+H]⁺—(15S)-(+)-6b), Rₜ=38.62 min. (m/z=497.4 [M+H]⁺—(15R)-(+)-6a).

### Isopropyl (5Z)-7-[(1R,2R,3R,5S)-3,5-Dihydroxy-2-[(E,3R)-3-hydroxy-4-[3-(trifluoromethy-lo)phenoxy]-1-butenyl]cydopentylc]-5-heptenate (travoprost) (1a)

To the solution of fluprostenol (15R)-(+)-6a (2.1 g, 4.58 mmol) in anhydrous acetone (20 ml) DBU (4.8 ml, 32.06 mmol) was added and after 5 min. 2-iodopropane (3.2 ml, 32.06 mmol) was added. The resulting mixture was stirred at ambient temperature for 26 h. The solution was diluted with ethyl acetate (150 ml) and the precipitated solid was filtered off and washed with ethyl acetate (4×25 ml). The combined organic phases were condensed under reduced pressure to approximately 50 ml volume, acidified to pH 5-6 with 3% aqueous solution of citric acid. Water phase was separated and extracted with ethyl acetate (3×25 ml). The combined organic extracts were washed with saturated NaHCO₃ solution (1×150 ml), brine (1×200 ml) and then dried over anhydrous Na₂SO₄ (20 g). The drying agent was filtered off and the filtrate was condensed under reduced pressure. The crude product (2.6 g) was purified by column chromatography (silica gel, 2-propanol/CH₂Cl₂ at 6%-10% concentration gradient) yielding (15R)-(+)-1a travoprost (2.10 g, 91.5%, 1a:1b:(5E,15R)-isomer=91.95%:0.18%:7.87%). [α]_{D}²⁰=+16.310 (c 1.0, CH₂Cl₂). (lit. [α]_{D}²⁰=+14.6° (c 1.0, CH₂Cl₂))². FT-IR (thin film) νₘₐₓ (cm⁻¹): 3376, 2975, 2934, 1727, 1592, 1493, 1450, 1375, 1241, 1168, 1127, 1066, 1035, 970, 951, 915, 882, 792, 698. ¹H NMR (C₆D₆, 600 MHz, 25° C.) δ (ppm): 1.05 (s, 3H, —CH₃), 1.06 (s, 3H, —CH₃), 1.36 (CH-1 of cyclopentyl ring), 1.63 (m, 2H, CH₂-3 of α chain), 1.88 (dd, J=4.2 i 15.0 Hz, one proton of CH₂-4 group of cyclopentyl ring), 2.06 (m, 1H, one proton of CH₂-4 group of α chain), 2.14-2.17 (m, 3H, CH₂-2 and one proton of CH₂-4 group of α chain), 2.22 (m, 2H, one proton of CH₂-7 group of α chain and one proton of CH₂-4 group of cyclopentyl ring), 2.47 (m, 1H, one proton of CH₂-7 group of α chain), 2.62 (m, 1H, CH-2 of cyclopentyl ring), 3.41 (s, 1H, OH), 3.80 (dd, J=4.2 i 9.6 Hz, one proton of CH₂-4 group of ω chain), 3.86 (dd, J=6.9 and 9.6 Hz, one proton of CH₂-4 group of ω chain), 3.96 (m, 1H, CH-3 of cyclopentyl ring), 4.08 (m, 1H, CH-5 of cyclopentyl ring), 4.30 (m, 1H, OH), 4.52 (m, 1H, CH-3 of ω chain), 4.98 (septet, 1H, —CH(CH₃)₂), 5.38 (m, 1H, CH-5 of α chain), 5.54 (m, 1H, CH-6 of α chain), 5.66 (dd, J=9.2 i 15.3 Hz, CH-1 of ω chain), 5.80 (dd, J=7.2 i 15.3 Hz, CH-2 of ω chain), 6.90 (dd, J=2.2 i 8.5 Hz, 1H, aromatic CH-6), 6.98 (m, 1H, aromatic CH-5), 7.05 (dd, J=7.7 Hz, 1H, aromatic CH-4), 7.25 (m, 1H, aromatic CH-2). ¹³C NMR (150 MHz, C₆D₆, 25° ° C.) δ (ppm): 21.74 (—CH₃), 21.76 (—CH₃), 25.19 (C-3 of α chain), 25.65 (C-7 of a α chain), 26.90 (C-4 of α chain), 34.05 (C-2 of α chain), 43.52 (C-4 of cyclopentyl ring), 50.18 (C-1 of cyclopentyl ring), 55.80 (C-2 of cyclopentyl ring), 67.61 (—CH(CH₃)₂), 71.45 (C-3 of ω chain), 72.16 (C-5 of cyclopentyl ring), 72.29 (C-4 of ω chain), 77.63 (C-3 of cyclopentyl ring), 111.87 (q, J=3.8 Hz, aromatic C-2), 117.73 (q, J=3.8 Hz, aromatic C-4), 118.44 (aromatic C-6), 124.80 (q, J=274.2 Hz, —CF₃), 129.61 (C-6 of α chain), 129.80 (C-5 of α chain), 130.33 (aromatic C-5), 131.38 (C-2 of ω chain), 132.0 (q, J=32.2 Hz, aromatic C-3), 136.0 (C-1 of ω chain), 159.39 (aromatic C-1), 173.30 (C═O). HRMS (ESI): calculated for C₂₆H₃₅O₆F₃Na [M+Na]⁺ 523.2278. found 523.2272.

HPLC: AS-3R, 3 μm, 150×4.6 mm, H₂O/CH₃CN, concentration gradient 80%-10%, 1.0 ml/min, Rₜ=32.670 min. (91.95% yield (15R)-(+)-1a), Rₜ=35.97 min. (0.18%—(15S)-(+)-1b), Rₜ=36.70 min. (7.87%—(5E,15R)-isomer).

HPLC-MS (ESI): AS-3R, 3 μm, 150×4.6 mm, H₂O/CH₃CN, concentration gradient 80%—10%, 1.0 ml/min, Rₜ=34.60 min. (m/z=501.3 [M+H]⁺—(15R)-(+)-1a), Rₜ=38.67 min. (m/z=501.3 [M+H]⁺—(15S+)(+)-1b), Rₜ=39.40 min. (m/z=501.3 [M+H]⁺—(5E,15R)-isomer).

### Izopropyl (5Z)-7-[(R,2R,3R,5S)-3,5-Dihydroxy-2-[(1E,3S)-3-hydroxy-4-[3-(trifluoromethyl)phenoxy]-1-butenyl]cyclopentyl]-5-heptenate (1b)

In the same manner, using (15S)-(+)-6b acid (1.80 g, 3.93 mmol), (15S)-(+)-1b ester was obtained, 1.80 g (91.4%, 1a:1b:(5E,15S)-isomer=0.53%:91.84%:7.63%). [α]_{D}²⁰=+27.15° (c 1.0, CH₂Cl₂). FT-IR (thin film) νₘₐₓ (cm⁻¹): 3394, 2981, 2936, 1726, 1593, 1493, 1450, 1330, 1241, 1168, 1127, 1109, 1066, 1036, 971, 917, 881, 792, 698. ¹H NMR (C₆D₆, 600 MHz, 25° C.) δ (ppm): 1.05 (s, 3H, —CH₃), 1.06 (s, 3H, —CH₃), 1.38 (m, 1H, CH-1 of cyclopentyl ring), 1.63 (m, 2H, CH₂-3 of α chain), 1.85 (m, 1H, one proton of CH₂-4 group of cyclopentyl ring), 2.09 (m, 2H, one proton of CH₂-4 group of cyclopentyl ring and one proton of CH₂-4 group of α chain), 2.14-2.19 (m, 3H, one proton of CH₂-4 and CH₂-2 group of α chain), 2.30 (m, 1H, one proton of CH₂-7 group of α chain), 2.45 (m, 1H, one proton of CH₂-7 group of α chain), 2.59 (m, 1H, CH-2 of cyclopentyl ring), 3.30 (br s, —OH), 3.73 (br s, —OH), 3.94 (br s, —OH), 3.79 (d, J=5.5 Hz, 2H, CH₂-4 of ω chain), 3.97 (s, 1H, CH-3 of cyclopentyl ring), 4.08 (m, 1H, CH-5 of cyclopentyl ring), 4.51 (m, 1H, CH-3 of ω chain), 4.99 (septet, 1H, —CH(CH₃)₂), 5.38 (m, 1H, CH-5 of α chain), 5.57 (m, 1H, CH-6 of α chain), 5.75-5.78 (m, 2H, CH-1 and CH-2 of ω chain), 6.87 (aromatic CH-6), 6.97 (aromatic CH-5), 7.04 (aromatic CH-4) and 7.23 (aromatic CH-2). ¹³C NMR (150 MHz, C₆D₆, 25° C.) δ (ppm): 21.74 (—CH₃), 21.75 (—CH₃), 25.2 (C-3 of α chain), 25.78 (C-7 of α chain), 26.89 (C-4 of α chain), 34.0 (C-2 of α chain), 43.62 (C-4 of cyclopentyl ring), 50.87 (C-1 of cyclopentyl ring), 55.80 (C-2 of cyclopentyl ring), 67.72 (—CH(CH₃)₂), 70.61 (C-3 of ω chain), 72.46 (C-4 of ω chain), 72.63 (C-5 of cyclopentyl ring), 78.05 (C-3 of cyclopentyl ring), 111.85 (q, J=4.0 Hz, aromatic C-2), 117.74 (q, J=3.6 Hz, aromatic C-4), 118.45 (aromatic C-6), 124.76 (q, J=272.3 Hz, —CF₃), 129.73 (C-6 of α chain), 129.75 (C-5 of α chain), 130.29 (aromatic C-5), 130.55 (C-2 of ω chain), 132.0 (q, J=32.2 Hz, aromatic C-3), 134.54 (C-1 of ω chain), 159.36 (aromatic C-1), 173.44 (C═O). HRMS (ESI): calculated for C₂₆H₃₅O₆F₃Na [M+Na]⁺ 523.2278. found 523.2287

HPLC: AS-3R, 3 μm, 150×4.6 mm, H₂O/CH₃CN, concentration gradient 80%-10%, 1.0 ml/min, Rₜ=32.67 min. (0.53%—(15R)-(+)-1a), Rₜ=35.97 min. (91.84%—(15S)-(+)-1b), Rₜ=39.43 min. (7.63%—(5E,15S)-isomer).

HPLC-MS (ESI): AS-3R, 3 μm, 150×4.6 mm, H₂O/CH₃CN, concentration gradient 80%—10%, 1.0 ml/min, Rₜ=34.60 min. (m/z=501.3 [M+H]⁺—(15R)-(+)-1a), Rₜ=38.67 min. (m/z=501.3 [M+H]⁺—(15S)-(+)-1b), Rₜ=41.86 min. (m/z=501.3 [M+H]⁺—(5E,15S)-isomer).

### Example 2

### Synthesis of (3S)-(+)-7a bimatoprost and its (3R)-(+)-(7b) epimer

### (R)-(−)-4-phenylbutane-1,2-diyl bis(4-nitrobenzoate) (21a)

DIAD (4.70 ml, 23.118 mmol) was added dropwise to a stirred solution of diol (S)-(−)-20a (1.50 g, 9.247 mmol), p-nitrobenzoic acid (3.90 g, 23.118 mmol) and PPh₃ (6.12 g, 23.118 mmol) in anhydrous toluene (50 ml) at 0° C. under an argon atmosphere. After stirring for 2 h at room temperature, TLC analysis (hexanes/AcOEt 4:1) indicated the reaction was complete. The excess of toluene (20 ml) was evaporated and the residue was put into refrigerator for several hours. Triphenylphosphine oxide was removed by filtration on a Büchner funnel and washed with cold toluene (3×15 ml). The filtrate and washings were combined and concentrated under reduced pressure to give the crude product (4.39 g) as an orange-yellow solid. Crystallization from a mixture of hexanes/AcOEt (1:1) afforded the pure diester (R)-(−)-21a (3.81 g, 88.7% yield) as a light yellow crystals. [α]_{D}²⁰=−6.75° (c 1.0, acetone) (lit.¹⁵ [α]_{D}²⁰=−6.0° (c 1.15, acetone)). mp 115.10-121.47° C., peak 117.14° C., heating rate 10.00° C./min (lit.¹⁵ mp 114-115° C.). FT-IR (KBr) νₘₐₓ (cm⁻¹): 3111, 3079, 2998, 2939, 2863, 1731, 1717, 1606, 1525, 1349, 1331, 1289, 1262, 1122, 1103, 1011, 875, 839, 784, 718, 758, 505. ¹H NMR (600 MHz, CDCl₃, 25° C.) δ (ppm): 2.16 (m, 1H, one of the CH₂-3 group), 2.25 (m, 1H, one of the CH₂-3 group), 2.82 (m, 2H, CH₂-4), 4.54 (dd, J=6.9 and 12.1 Hz, 1H, one of the CH₂-1 group), 4.67 (dd, J=3.2 and 12.1 Hz, 1H, one of the CH₂-1 group), 5.58 (m, 1H, CH-2), 7.19 (m, 1H, H-4 of the phenyl ring), 7.20 (m, 2H, H-2 and H-6 of the phenyl ring), 7.28 (m, 2H, H-3 and H-5 of the phenyl ring), 8.15 (m, 4H, H-2 and H-6 of the aryl rings), 8.27 (m, 4H, H-3 and H-5 of the aryl rings). ¹³C NMR (150 MHz, CDCl₃, 25° C.) δ (ppm): 31.57 (C-4), 32.32 (C-3), 66.25 (C−1), 72.74 (C-2), 126.29 (C-4 of the phenyl ring), 128.26 (C-2 of the phenyl ring), 128.65 (2C, C-3 and C-5 of the phenyl ring), 140.29 (C-1 of the phenyl ring), 123.63 (4C, C-3 and C-5 of the aryl rings), 130.75 (4C, C-2 and C-6 of the aryl rings), 135.00 (2C, C-1 of the aryl rings), 150.7 (2C, C-4 of the aryl rings), 164.20 (2-C(O)OPhNO₂), 164.29 (1-C(O)OPhNO₂). HRMS (ESI): calcd. for C₂₄H₂₀N₂O₈Na [M+Na]⁺ 487.1112; fund 487.1129.

### (R)-(+)-4-Phenyl-1,2-butanediol (20b)

The diester (R)-(−)-21a (3.65 g, 7.856 mmol) was added to a suspension of LiOH.H₂O (1.65 g, 39.28 mmol) in MeOH (25 ml). The reaction mixture was stirred for 5 h at room temperature. TLC analysis (CH₂Cl₂/MeOH 19/1) indicated disappearance of the starting diester (R)-(−)-21a. After MeOH evaporation, the residual solid was dissolved in water (150 ml) and the product was extracted with tert-butyl methyl ether (3×25 ml). The combined ethereal solution was washed with brine (150 ml) and dried over anhydrous Na₂SO₄. Filtration and evaporation in vacuo gave the crude product (1.40 g), which was purified by flash column chromatography over silica gel (CH₂Cl₂/MeOH 19/1) to afford the pure diol (R)-(+)-20b (1.27 g, 97.4%, 99.50% ee) as a white solid. [α]_{D}²⁰=+34.21° (c 1.0, EtOH) (lit.¹⁵ [α]_{D}²⁰=+33° (c 1.88, EtOH)). mp 36.49-42.36° C., peak 39.23° C., heating rate 10.00° C./min. FT-IR (KBr) νₘₐₓ (cm⁻¹): 3334, 3262, 3024, 3003, 2945, 2926, 2857, 1946, 1877, 1603, 1491, 1466, 1452, 1414, 1307, 1262, 1197, 1104, 1037, 1014936, 913, 864, 749, 700, 668, 591, 527, 508. ¹H NMR (600 MHz, CDCl₃, 25° C.) δ (ppm): 1.73 (m, 2H, CH₂-3), 2.67 (ddd, J=7.7, 9.2 and 13.8 Hz, 1H, one of the CH₂-4 group), 2.78 (ddd, J=5.5, 9.2, and 14.0 Hz, one of the CH₂-4 group), 3.10 (br. s, 2H, two —OH groups), 3.44 (m, 1H, CH-2), 3.62 (m, 1H, one of the CH₂-1 group), 3.70 (m, 1H, one of the CH₂-1 group), 7.18 (m, 2H, aromatic H-2 and H-6), 7.19 (m, 1H, aromatic H-4) and 7.28 (m, 2H, aromatic H-3 and H-5). ¹³C NMR (150 MHz, CDCl₃, 25° C.) δ (ppm): 31.78 (C-4), 34.62 (C-3), 66.7 (C−1), 71.57 (C-2), 125.95 (aromatic C-4), 128.39 (2C, aromatic C-3 and C-5), 128.44 (2C, aromatic C-2 and C-6), 141.68 (aromatic C-1). HRMS (EI-HR): calcd. for C₁₀H₁₄O₂ 166.09938; fund 166.10008.

HPLC: Chiracel OD OD00CE-EL068, 10 μm, 250×4.6 mm column, hexanes/2-propanol 9:1 (v/v), 1.0 ml/min, Rₜ 13.184 min. (99.75% yield of 20b), Rₜ=19.040 (0.25% yield of 20a), 99.50% ee.

### (S)-(−)-2-Hydroxy-4-phenylbutyl pivalate (22a)

Trimethylacetyl chloride (1.57 ml, 12.634 mmol) was added to a stirred solution of diol (S)-(−)-21a (2.00 g, 12.032 mmol) in a mixture of CH₂Cl₂ and pyridine (1:1, 50 ml) at 0° C. under an argon atmosphere. After stirring at 0° C. for 1 h and at room temperature for 1 h, the reaction was quenched with crushed ice (25 g) and the whole was portioned between AcOEt (25 ml) and 10% aqueous HCl (25 ml). The resulting layers were separated and the aqueous phase was extracted with AcOEt (3×25 ml). The combined organic extracts were washed successively with H₂O (150 ml), saturated aqueous NaHCO₃ (150 ml), brine (200 ml) and dried over anhydrous Na₂SO₄. Filtration and evaporation in vacuo furnished the crude ester (3.27 g), which was purified by flash column chromatography over silica gel (hexanes/AcOEt 4:1) to afford the pivalate (S)-(−)-22a (2.83 g, 93.9% yield, 99.64% ee) as a white solid. [α]_{D}²⁰=−19.97 (c 1.0, EtOH). mp 44.55-50.24° C., peak 47.02° C., heating rate 10.00° C./min. FT-IR (KBr) νₘₐₓ (cm⁻¹): 3542, 3400, 3082, 3027, 2971, 2949, 2916, 1707, 1478, 1457, 1367, 1280, 1175, 1070, 1036, 917, 744, 700. ¹H NMR (600 MHz, CDCl₃, 25° C.) δ (ppm): 1.22 (s, 9H, —C(CH₃)₃), 1.80 (m, 2H, CH₂-3), 2.16 (s, 1H, —OH), 2.71 (m, 1H, one of the CH₂-4 group), 2.82 (m, 1H, one of the CH₂-4 group), 3.84 (m, 1H, CH-2), 4.02 (dd, J=6.7 and 11.4 Hz, 1H, one of the CH₂-1 group), 4.12 (dd, J=3.4 and 11.4 Hz, 1H, one of the CH₂-1 group), 7.18 (m, 1H, aromatic H-4), 7.20 (m, 2H, aromatic H-2 and H-6), 7.28 (m, 2H, aromatic H-3 and H-5). ¹³C NMR (150 MHz, CDCl₃, 25° C.) δ (ppm): 27.20 (3C, —C(CH₃)₃), 31.58 (C-4), 34.96 (C-3), 38.88 (—C(CH₃)₃), 68.50 (C-1), 69.33 (C-2), 125.97 (aromatic C-4), 128.41 (2C, aromatic C-2 and C-6), 128.45 (2C, aromatic C-3 and C-5), 141.55 (aromatic C-1), 178.75 ((CH₃)₃C—C(O)O—). HRMS (ESI): calcd. for C₁₅H₂₂N₂O₃Na [M+Na]⁺ 273.14612; fund 273.1451.

HPLC: Chiracel OD-H, 5 μm, 250×4.6 mm column, hexanes/ethanol/2-propanol 100:1:1 (v/v/v), 0.7 ml/min, Rₜ 22.779 min. (99.82% yield of 22a), Rₜ=34.108 (0.18% yield of 22b), 99.64% ee.

### (R)-(+)-2-Hydroxy-4-phenylbutyl pivalate (22b)

According to the procedure described for the preparation of (S)-(−)-22a, the diol (R)-(+)-20b (1.20 g, 7.219 mmol) afforded the pivalate (R)-(+)-22b (1.65 g, 91.5% yield, 99.50% ee). [α]_{D}²⁰=+20.16° (c 1.0, EtOH). The characterization data from IR, NMR and HRMS spectra were identical in all aspects with those of (S)-(−)-22a enantiomer.

### (S)-(+)-2-(tert-Butyldimethylsilyloxy)-4-phenylbutyl pivalate (23a)

tert-Butyldimethylsilyl chloride (1.96 g, 12.607 mmol) was added in one portion to a stirred solution of alcohol (S)-(−)-22a (2.63 g, 10.506 mmol) and imidazole (1.16 g, 16.81 mmol) in anhydrous DMF (25 ml) at 0° C. under an argon atmosphere. The reaction was allowed to proceed for 18 h at room temperature and then quenched with crushed ice (25 g). The resulting mixture was portioned between hexanes (25 ml) and H₂O (50 ml). The aqueous layer was extracted with hexanes (3×25 ml). The combined organic extracts were washed successively with H₂O (100 ml), brine (150 ml) and dried over Na₂SO₄. Filtration and evaporation in vacuo furnished the crude product (3.97 g) as a light yellow oil, which was purified by flash column chromatography (silica gel, 20:1→10:1 hexanes/AcOEt) to give TBDMS ether (S)-(+)-23a (3.64 g, 95.1% yield) as a colourless oil. [α]_{D}²⁰=+3.98° (c 1.0, EtOH). FT-IR (thin film) νₘₐₓ (cm⁻¹): 3063, 3027, 2956, 2930, 2857, 1732, 1496, 1472, 1462, 1397, 1362, 1283, 1256, 1155, 1120, 1064, 1004, 836, 776, 699. ¹H NMR (600 MHz, CDCl₃, 25° C.) δ (ppm): 0.09 (s, 6H, (CH₃)₂Si), 0.91 (s, 9H, (CH₃)₃C—Si), 1.21 (s, 9H, (CH₃)₃C—C), 1.82 (m, 2H, CH₂-3), 2.64 (ddd, J=5.8, 10.8 and 13.8 Hz, 1H, one of the CH₂-4 group), 2.72 (ddd, J=5.8, 10.8 and 13.8 Hz, 1H, one of the CH₂-4 group), 3.91 (m, 1H, CH-2), 4.02 (m, 2H, CH₂-3), 7.18 (m, 2H, aromatic H-2 and H-6), 7.19 (m, 1H, aromatic H-4), 7.28 (m, 2H, aromatic H-3 and H-5). ¹³C NMR (150 MHz, CDCl₃, 25° C.) θ (ppm): −4.67 (CH₃—Si), −4.48 (CH₃—Si), 18.03 ((CH₃)₃C—Si), 25.78 (3C, (CH₃)₃C—Si), 27.23 (3C, (CH₃)₃C—C), 31.25 (C-4), 36.53 (C-3), 38.77 ((CH₃)₃C—C), 67.72 (C-1), 69.61 (C-2), 125.80 (aromatic C-4), 128.29 (2C, aromatic C-2 and C-6), 128.39 (2C, aromatic C-3 and C-5), 142.17 (aromatic C-1), 178.44 ((CH₃)₃CC(O)O—). HRMS (ESI): calcd. for C₂₁H₃₆O₃NaSi [M+Na]⁺ 387.2326; fund 387.2326.

### (R)-(−)-2-(tert-Butyldimethylsilyloxy)-4-phenylbutyl pivalate (23b)

In the same manner as described for the preparation of (S)-(+)-23a, the alcohol (R)-(+)-22b (1.5 g, 5.992 mmol) afforded the ether (R)-(−)-23b (2.05 g, 93.7% yield) as a colourless oil. [α]_{D}²⁰=−3.48° (c 1.0, EtOH). The characterization data from IR, NMR and HRMS spectra were identical in all aspects with those of(S)-(+)-23a enantiomer.

### (S)-(−)-2-(tert-butyldimethylsilyloxy)-4-phenylbutan-1-ol (24a)

Diisobutylaluminum hydride in toluene (1.0 M, 24 ml, 24.00 mmol) was added dropwise over 15 min. to a stirred solution of pivalate (S)-(+)-23a (3.45 g, 9.463 mmol) in anhydrous CH₂Cl₂ (50 ml) at −78° C. under an argon atmosphere. The resulting mixture was allowed to warm to ×20° C. for a 30 min period and stirred at this temperature for another 2 h. TLC analysis (hexanes/AcOEt 8:1) indicated disappearance of the starting pivalate (S)-(+)-23a. The clear colourless solution was re-cooled to −78° C. and the excess of DIBAL was quenched by addition of MeOH (15 ml) dropwise. On warming to 0° C., 10% aqueous potassium sodium tartrate (100 ml) was added and the mixture was stirred vigorously at room temperature for 2 h. The resulting layers were separated and the aqueous layer was extracted with CH₂Cl₂ (3×25 ml). The combined extracts were washed with water (100 ml), brine (150 ml) and dried over anhydrous Na₂SO₄. Filtration and evaporation in vacuo furnished the crude product (2.89 g), which was purified by flash column chromatography (silica gel, 3%-9% hexanes/AcOEt) to afford the primary alcohol (S)-(−)-24a (2.51 g, 94.6% yield, 99.68% ee) as a colourless oil. [α]_{D}²⁰=−12.69° (c 1.0, EtOH). FTIR (thin film) νₘₐₓ (cm⁻¹): 3420, 3063, 3026, 2953, 2929, 2857, 1469, 1496, 1472, 1462, 1388, 1361, 1255, 1113, 1045, 988, 836, 776, 698, 665. ¹H NMR (600 MHz, CDCl₃, 25° C.) δ (ppm): 0.08 (s, 3H, CH₃—Si), 0.09 (s, 3H, CH₃—Si), 0.91 (s, 9H, (CH₃)₃C—Si), 1.83 (m, 2H, CH₂-3), 1.85 (m, 1H, —OH), 2.64 (m, 2H, CH₂-4), 3.52 (dd, J=5.2 and 11.1 Hz, 1H, one of the CH₂-1 group), 3.60 (dd, J=3.8 and 11.1 Hz, 1H, one of the CH₂-1 group), 3.79 (m, 1H, CH-2), 7.18 (m, 2H, aromatic H-2 and H-6), 7.19 (m, 1H, aromatic H-4), 7.28 (m, 2H, aromatic H-3 and H-5). ¹³C NMR (150 MHz, CDCl₃, 25° C.) δ (ppm): −4.49 (CH₃—Si), −4.52 (CH₃—Si), 18.10 ((CH₃)₃C-Si), 25.85 (3C, (CH₃)₃C—Si), 31.71 (C-4), 35.71 (C-3), 66.14 (C-1), 72.43 (C-2), 125.84 (aromatic C-4), 128.26 (2C, aromatic C-2 and C-6), 128.39 (2C, aromatic C-3 and C-5), 142.01 (aromatic C-1). HRMS (ESI): calcd. for C₁₆H₂₈O₂NaSi [M+Na]⁺303.17508; fund 303.1750.

HPLC: Chiracel OD-H, 5 μm, 250×4.6 mm column, hexanes/ethanol/methanol 98:1.5:0.5 (v/v/v), 1.0 ml/min, Rₜ 8.607 min. (99.84% yield of 24a), R₄=10.846 (0.16% yield of 24b), 99.68% ee.

### (R)-(+)-2-(tert-butyldimethylsilyloxy)-4-phenylbutan-1-ol (24b)

Treatment of the pivalate (R)-(−)-23b (1.88 g, 5.156 mmol) similar to the reduction of(S)-(+)-23a afforded the primary alcohol (R)-(+)-24b (1.35 g, 93.3% yield, 99.50% ee) as a colourless oil. [α]_{D}²⁰=+12.25° (c 1.0, EtOH). The characterization data from IR, NMR and HRMS spectra were identical in all aspects with those of (S)-(−)-24a enantiomer.

### (S)-(−)-2-(tert-butyldimethylsilyloxy)-4-phenylbutanal (16a)

### Method A.

Dess-Martin periodinane (4.45 g, 10.182 mmol) was added portionwise to a cold (0° C.) suspension of alcohol (S)-(−)-24a (2.38 g, 8.485 mmol) and dry NaHCO₃ (2.14 g, 25.455 mmol) in anhydrous CH₂Cl₂ (50 ml). After being stirred for 1 h at room temperature, TLC analysis (hexanes/AcOEt 10:1) indicated disappearance of the starting alcohol (S)-(−)-24a. Saturated aqueous NaHCO₃ (100 ml) and Na₂SO₃ (7.49 g, 59.395 mmol) were then added simultaneously and the mixture was stirred at room temperature for 30 min. The resulting layers were separated and the aqueous phase was extracted with CH₂Cl₂ (3×25 ml). The combined extracts were washed with brine (3×150 ml) and dried over Na₂SO₄. Filtration and evaporation in vacuo furnished the crude product (2.34 g), which was purified by flash column chromatography (silica gel, 10:1 hexanes/AcOEt) to afford the aldehyde (S)-(−)-16a (2.22 g, 94.2%) as a colourless oil. [α]_{D}²⁰=−19.77° (c 1.0, CHCl₃). FT-IR (thin film) νₘₐₓ (cm⁻¹): 3063, 3028, 2954, 2929, 2799, 2857, 1736, 1497, 1472, 1463, 1361, 1255, 1116, 1006, 973, 838, 778, 747, 699, 670. ¹H NMR (CDCl₃, 600 MHz, 25° C.) δ (ppm): 0.086 (s, 3H, CH₃—Si), 0.088 (s, 3H, CH₃—Si), 0.95 (s, 9H, (CH₃)₃C—Si), 1.96 (m, 2H, CH₂-3), 2.71 (m, 2H, CH₂-4), (4.02 (ddd, J=1.5, 5.3 and 6.8 Hz, 1H, CH-2], 7.18 (m, 2H, aromatic H-2 and H-6), 7.19 (m, 1H, aromatic H-4), 7.28 (m, 2H, aromatic H-3 and H-5), 9.59 (d, J=1.5 Hz, 1H, —CHO). ¹³C NMR (CDCl₃, 150 MHz, 25° C.) δ (ppm): −4.58 (CH₃—Si), —4.91 (CH₃—Si), 18.19 ((CH₃)₃C—Si), 25.75 (3C, (CH₃)₃C—Si), 30.80 (C-4), 34.53 (C-3), 77.11 (C-2), 126.09 (aromatic C-4), 128.42 (2C, aromatic C-2 and C-6), 128.46 (2C, aromatic C-3 and C-5), 141.22 (aromatic C-1), 204.08 (—CHO).

### Method B.

Sulfur trioxide pyridine complex (9.381 g, 57.759 mmol) was added over 15 min to a stirred solution of alcohol (S)-(−)-24a (5.4 g, 19.253 mmol) and Et₃N (16.35 ml, 115.518 mmol) in anhydrous DMSO (90 ml) under an argon atmosphere. After stirring for 1 h, the mixture was diluted with CH₂Cl₂ (180 ml) and poured into saturated aqueous NH₄Cl (180 ml) at 0° C. The resulting layers were separated and the aqueous phase was extracted with CH₂Cl₂ (3×50 ml). The combined organic extracts were washed successively with H₂O (150 ml), brine (150 ml) and dried over anhydrous Na₂SO₄. Filtration and evaporation in vacuo furnished the crude product (5.34 g), which was purified by flash column chromatography over silica gel (hexanes/AcOEt 10:1) to afford the aldehyde (S)-(−)-16a (4.503 g, 84.0%) as a colourless oil. [α]_{D}²⁰=−19.24° (c 1.0, CHCl₃). The characterization data from IR and NMR spectra were identical in all aspects with those of (S)-(−)-16a obtained according to the Method A.

### (R)-(+)-2-(tert-butyldimethylsilyloxy)-4-phenylbutanal (16b)

According to the procedure described for the preparation of (S)-(−)-16a (Method A), the alcohol (R)-(+)-24b (1.20 g, 4.278 mmol) yielded the aldehyde (R)-(+)-16b (1.12 g, 93.6% yield) as a colourless oil. [α]_{D}²⁰=+20.45° (c 1.0, CHCl₃). The characterization data from IR and NMR spectra were identical in all aspects with those of (S)-(−)-16a enantiomer.

### 1-[(4Z)-6-[(1R,2R,3R,5)-2-[(1R/1S,2R/2S,3S)-3-(tert-Buthyldimethylsilyiloxy)-5-phe-nyl-1-(phenylsulfonyl)-1-pentyl]-3,5-bis(triethylsilyloxy)cyclopentyl]-4-hexenyl]-4-methyl-2,6,7-trioxabicyclo[2.2.2]octan (9a)

To the solution of diisopropylamine (8.0 ml, 56.34 mmol) in anhydrous THF (40 ml) cooled to −60° C., n-BuLi (33.0 ml, 52.80 mmol, 1.6 M in hexane) was added dropwise, followed by addition of LA-5 sulfone (26.50 g, 38.12 mmol, 83.1% de) in anhydrous THF (40 ml) under argon atmosphere. The obtained mixture was stirred at −60° C. for 30 min. and the solution of (S)-(−)-8a aldehyde (13.4 g, 48.12 mmol) in anhydrous THF (10 ml) was added. After 20 min. the cooling bath was removed and brine (30 ml) was added. When two layers were separated, water phase was extracted with THF (3×50 ml). The combined organic layers were dried over anhydrous Na₂SO₄ (20 g). The drying agent was filtered off and the filtrate was condensed under vacuum. The crude diastereoisomeric mixture of (15S)-9a hydroxysulfones was obtained in 39.62 g yield, it was used in the next steps without purification.

### 1-[(4Z)-6-[(1R,2R,3R,5S)-2-[(1R/1S,2R/2S,3R)-3-(tert-Butylodimethylsilyloxy)-5-phe-nyl-1-(phenylsulfonyl)-1-pentyl]-3,5-bis(triethylsilyloxy)cyclopentyl]-4-hexenyl]-4-methyl-2,6,7-trioxabicyclo[2.2.2]octan (9b)

In the same manner, using LA-5 sulfone (28.0 g, 40.28 mmol, 83.1% de) and (R)-(+)-8b aldehyde (13.8 g, 49.56 mmol), the crude diastereoisomeric mixture of (15S)-9b hydroxysulfones in 41.72 g yield was obtained.

### 1-[(4Z)-6-[(1R,2R,3R,5S)-2-[(1E,3S)-3-(tert-Buthyldimethylsilyloxy)-5-phenyl-1-pen-tenyl]-3,5-bis(triethylsilyloxy)cydopentyl]-4-hexenyl]-4-methyl-2,6,7-trioxa-bicyclo[2.2.2]octan (10a)

The solution of (15S)-9a hydroxysulfones crude mixture (39.62 g) in THF (30 ml) was treated with saturated methanolic solution of Na₂HPO₄ (200 ml) and next, sodium amalgam (20 g, 173.99 mmol Na, 20%) was added portionwise during 4 h. Stirring was continued for 16 h. The solution was decantated over the amalgam and condensed under reduced pressure. The residue was diluted with water (250 ml) and ethyl acetate (150 ml). The layers were separated, water phase was extracted with ethyl acetate (3×100 ml). The combined organic extracts were dried over anhydrous Na₂SO₄ (20 g). The drying agent was filtered off and the filtrate was condensed under vacuum. (15S)-10a Olefin (31.76 g) was obtained, which was used in the next steps without purification.

### 1-[(4Z)-6-[(1R,2R,3R,5S)-2-[(1E,3R)-3-(tert-Butyldimethylsilyloxy)-5-phenyl-1-pentenyl]-3,5-bis(triethylsilyloxy)cydopentyl]-4-hexenyl]-4-methyl-2,6,7-trioxa-bicyclo[2.2.2]octan (10b)

Following the same procedure, using (15S)-9b hydroxysulfones crude mixture (41.72 g), 33.47 g of crude (15S)-10b olefin was obtained.

### 2,2-bis(Hydroxymethyl)propyl (5Z)-7-[(1R,2R,3R,5S)-3,5-Dihydroxy-2-[(1E,3S)-3-hydroxy-5-phenyl-1-pentenyl]-cyclopentyl]-5-heptenate (11a)

Tetrabutylammonium fluoride (115.0 ml, 115.0 mmol, 1.0 M in THF) was added dropwise to the solution of crude prostaglandin silyl derivative (15S)-10a (31.76 g) in anhydrous THF (100 ml). The resulting mixture was heated at 60° C. for 2 h. When the reaction was completed the solvent was evaporated and the oily residue was diluted with 10% aqueous solution of citric acid (200 ml) to remove 4-methyl-OBO protecting group. After 15 min. the reaction product was salted out with sodium chloride, separated and dried under reduced pressure. The crude product (20.23 g) was purified by column chromatography (silica gel, methanol/ethyl acetate at concentration gradient from 2% to 6%) yielding pentaol (15S)-(+)-11a (16.42 g, 87.8% yield from LA-5, 11a:11b:(5E,15S)-isomer=91.42%:0.17%:8.41%). [α]_{D}²⁰=+29.58° (c 1.0, CHCl₃). FT-IR (thin film) νₘₐₓ (cm⁻¹): 3373, 3024, 2932, 2837, 1732, 1603, 1496, 1454, 1374, 1246, 1172, 1047, 972, 923, 748, 701, 609. ¹H NMR (CDCl₃, 600 MHz, 25° C.) δ (ppm): 0.84 (s, 3H, —CH₃), 1.48 (m, 1H, of cyclopentyl ring CH-1), 1.67-1.70 (m, 3H, CH₂-3 of α chain and one proton of CH₂-4 group of cyclopentyl ring), 1.80 (m, 1H, one proton of CH₂-4 group of ω chain), 1.90 (m, 1H, one proton of CH₂-4 group of ω chain), 2.06-2.09 (m, 2H, one proton of CH₂-7 group and one proton of CH₂-4 group of α chain), 2.12-2.26 (m, 3H, one proton of CH₂-4 group of cyclopentyl ring, one proton of CH₂-4 group and one proton of CH₂-7 group of α chain), 2.32 (m, 1H, CH-2 of cyclopentyl ring), 2.34 (m, 2H, CH₂-2 of α chain), 2.67 (m, 2H, CH₂-5 of ω chain), 3.52 (m, 4H, two —CH₂OH groups), 3.89 (m, 1H, CH-3 of cyclopentyl ring), 4.06 (m, 1H, CH-3 of ω chain), 4.08 (m, 2H, CH₂-1 of α chain), 4.11 (m, 1H, CH-5 of cyclopentyl ring), 5.34 (m, 1H, CH-5 of α chain), 5.42 (m, 1H, CH-6 of α chain), 5.45 (dd, 1H, J=9.0 Hz i 15.29 Hz, CH-1 of ω chain), 5.58 (dd, J=7.5 Hz i 15.2 Hz, CH-2 of a chain), 7.17 (m, 1H, aromatic H-4), 7.19 (m, 2H, aromatic H-2 i H-6), 7.26 (m, 2H, aromatic H-3 i H-5). ¹³C NMR (150 MHz, CDCl₃, 25° C.) δ (ppm): 16.82 (—CH₃), 24.66 (C-3 of α chain), 25.57 (C-7 of α chain), 26.48 (C-4 of α chain), 31.81 (C-5 of ω chain), 33.45 (C-2 of α chain), 38.66 (C-4 of ω chain), 40.53 (—C(CH₃)(CH₂OH)₂), 42.78 (C-4 of cyclopentyl ring), 49.47 (C-1 of cyclopentyl ring), 55.42 (C-2 cyclopentyl ring), 66.51 (—CH₂C(CH₃)(CH₂OH)₂), 66.58 (2C, two —CH₂OH groups), 72.38 (C-3 of ω chain), 72.45 (C-5 cyclopentyl ring), 77.54 (C-3 of cyclopentyl ring), 125.80 (C-4 aromatic), 128.36 (2C, C-3 and C-5 aromatic), 128.5 (2C, C-2 and C-6 aromatic), 129.31 (C-6 of α chain), 129.44 (C-5 of α chain), 133.29 (C-1 of ω chain), 135.16 (C-2 of ac chain), 141.89 (C-1 aromatic), 174.61 (C═O). HRMS (ESI): calculated for C₂₈H₄₂O₇Na [M+Na]⁺ 513.28228. found 513.2837.

HPLC: Chiralpak OD-3R, 3 μm, 150×4.6 mm, H₂O/TEA (1000/1, adjusted to pH=4.5 with H₃PO₄ (phase A)/CH₃CN (phase B), concentration gradient 80%-10%, 1.0 ml/min, Rₜ=25.01 min. (91.42%—(15S)-(+)-1a), Rₜ=26.91 min. (8.41%—(5E,15S)-isomer) Rₜ=27.68 min. (0.17%—(15R)-(+)-11b).

HPLC-MS (ESI): Chiralpak OD-3R, 3 μm, 150×4.6 mm, H₂O/TEA (1000/1, adjusted to pH=4.5 with H₃PO₄ (phase A)/CH₃CN (phase B), concentration gradient 80%-10%, 1.0 ml/min., Rₜ=26.55 min. (m/z=490.2 [M+H]⁺—(15S)-(+)-11a), R_{t=28.34} min. (m/z=490.2 [M+H]⁺—(5E,15S)-isomer), Rₜ=30.03 min. (m/z=490.2 [M+H]⁺—(15R)-(+)-11b).

### 2,2-bis(Hydroxymethyl)propyl (Z)-7-[(1R,2R,3R,5S)-3,5-dihydroxy-2-[(1E,3R)-3-hydroxy-5-phenyl-1-pentenyl]-cyclopentyl]-5-heptenate (11b)

In the same manner, using crude olefin (15S)-10a (33.47 g), 17.06 g of 3R)-(+)-11b pentaol was obtained ((86.3% yield from LA-5, 11a:11b:(5E,15R)-isomer=0.27%:91.34%:8.39%). [α]_{D}²⁰=+23.70° (c 1.0, CHCl₃). FT-IR (thin film) νₘₐₓ (cm⁻¹): 3363, 3061, 3024, 2933, 2847, 1715, 1603, 1496, 1454, 1247, 1173, 1031, 972, 923, 748, 701, 588. ¹H NMR (CDCl₃, 600 MHz, 25° C.) δ (ppm): 0.83 (s, 3H, —CH₃), 1.47 (m, 1H, C-1 of cyklopentyl ring), 1.66-1.70 (m, 3H, CH₂-3 of α chain and one proton of CH₂-4 group of cyclopentyl ring), 1.85 (m, 2H, CH₂-4 of ω chain), 2.09-2.11 (m, 3H, CH₂-4 and one proton of CH₂-7 group of α chain), 2.18-2.23 (m, 2H, one proton of CH₂-7 group of α chain chain one proton of CH₂-4 group of cyclopentyl ring), 2.30 (m, 1H, CH-2 of cyclopentyl ring), 2.32 (m, 2H, CH₂-2 of α chain), 2.66 (m, 1H, CH-5 of ω chain), 2.72 (m, 1H, CH-5 of ω chain), 3.50 (m, 4H, two —CH₂OH groups), 3.91 (m, 1H, CH-3 of cyclopentyl ring), 4.06 (m, 2H, CH₂-1 of α chain), 4.08 (m, 1H, CH-3 of ω chain), 4.11 (m, 1H, CH-5 of cyclopentyl ring), 5.33 (m, 1H, CH-5 of α chain), 5.43 (m, 1H, CH-6 of α chain), 5.52 (dd, 1H, J=8.7 Hz and 15.4 Hz, CH-1 of ω chain), 5.62 (dd, J=6.0 Hz and 15.4 Hz, 1H, CH-2 of ω chain), 7.16 (m, 1H, H-4 aromatic), 7.17 (m, 2H, H-2 and H-6 aromatic), 7.26 (m, 2H, H-3 and H-5 aromatic). ¹³C NMR (150 MHz, CDCl₃, 25° C.) δ (ppm): 16.75 (—CH₃), 24.59 (C-3 of a chain), 25.50 (C-7 of α chain), 26.39 (C-4 of α chain), 31.76 (C-5 of ac chain), 33.35 (C-2 of α chain), 38.60 (C-4 of α chain), 40.48 (—C(CH₃)(CH₂OH)₂), 42.87 (C-4 of cyclopentyl ring), 50.17 (C-1 of cyclopentyl ring), 55.13 (C-2 of cyclopentyl ring), 66.35 (2C, two —CH₂OH groups), 66.54 (—CH₂C(CH₃)(CH₂OH)₂), 71.47 (C-3 of ω chain), 72.55 (C-5 of cyclopentyl ring), 77.63 (C-3 of cyclopentyl ring), 125.75 (C-4 aromatic), 128.32 (2C, C-3 and C-5 aromatic), 128.39 (2C, C-2 and C-6 aromatic), 129.32 (C-5 of α chain), 129.35 (C-6 of α chain), 131.95 (C-1 of ω chain), 134.58 (C-2 of ω chain), 141.92 (C-1 aromatic), 174.64 (C═O). HRMS (ESI): calculated for C₂₈H₄₂O₇Na [M+Na]⁺ 513.28228. found 513.2829.

Chiral HPLC: Chiralpak OD-3R, 3 μm, 150×4.6 mm, H₂O/TEA (1000/1, adjusted to pH=4.5 with H₃PO₄ (phase A)/CH₃CN (phase B), concentration gradient 80%-10%, 1.0 ml/min, Rₜ=25.01 min. (0.27%—(15S-(−)-11a), Rₜ=27.68 min. (91.34%—(15R)-(+)-11b), Rₜ=30.19 min. (8.39%—(5E,15R)-isomer).

HPLC-MS (ESI): Chiralpak OD-3R, 3 μm, 150×4.6 mm, H₂O/TEA (1000/1, adjusted to pH=4.5 with H₃PO₄ (phase A)/CH₃CN (phase B), concentration gradient 80%-10% h, 1.0 ml/min, Rₜ=26.55 min. (m/z=490.2 [M+H]⁺—(15S)-(+)-26a), Rₜ=30.03 min. (m/z=490.2 [M+H]⁺—(15R)-(+)-26b), Rₜ=31.58 min. (m/z=490.2 [M+H]⁺—(5E,15R)-isomer).

### (5Z)—N-ethyl-7-[(1R,2R,3R,5S)-3,5-Dihydroxy-2-[(1E,3S)-3-hydroxy-5-phenyl-1-pentenyl]-cyclopentyl]hept-5-enamid (7a)

(15S)-(+)-11a Pentaol (11.0 g, 22.42 mmol) was dissolved in 70% aqueous solution of EtNH₂ (50 ml). The mixture was stirred at ambient temperature for 72 h. When the reaction was completed (TLC, methanol/methylene dichloride 10%), the solution was condensed under reduced pressure. The oily residue was diluted with brine (40 ml) and ethyl acetate (40 ml), the layers were separated and organic phase was extracted with ethyl acetate (3×40 ml). The combined organic layers were dried over anhydrous Na₂SO₄ (20 g). The drying agent was filtered off and the filtrate was condensed under reduced pressure. The crude product (9.72 g) was purified by column chromatography (silica gel, methanol/ethyl acetate in concentration gradient from 5% to 10%) yielding (15S)-(+)-7a bimatoprost (8.12 g, 87.2%, 7a:7b:(5E,15S)-isomer=91.50%:0.12%:8.38%) as a pale yellow oil. The oily product was macerated with tert-buthylmethyl ether (50 ml), precipitated solid was filtered and recrystallized (ethyl acetate/tert-buthylmethyl ether) resulting in (15S)-(+)-7a bimatoprost of pharmaceutical purity (6.80 g, 83.7% yield, HPLC purity 99.34%, 7a: (5E,5S)-isomer=99.36%:0.64%, 98.72% de) as white solid. [α]_{D}²⁰=+39.07° (c 1.0, CH₂Cl₂). (lit.³ [α]_{D}²⁰=+32.7 (c 0.33, CH₂Cl₂)). melting point 65.70-72.70° C., maximum 69.52° C., temp. increase 10.00° C./min (lit.² mp 67-68° C.). FT-IR (KBr) νₘₐₓ (cm⁻¹): 3420, 3327, 3084, 3011, 2914, 2865, 2933, 1620, 1546, 1496, 1456, 1372, 1317, 1290, 1249, 1151, 1097, 1055, 1027, 976, 920, 698. ¹H NMR (CDCl₃, 600 MHz, 25° C.) δ (ppm): 1.10 (t, J=7.2 Hz, 3H, —CH₂CH₃), 1.46 (m, 1H, CH-1 of cyclopentyl ring), 1.62 (m, 1H, one proton of CH₂-3 group of α chain), 1.68 (m, 1H, one proton of CH₂-3 group of α chain), 1.74 (m, 1H, one proton of CH₂-4 group of cyclopentyl ring), 1.78 (m, 1H, one proton of CH₂-4 group of ca chain), 1.90 (m, 1H, one proton of CH₂-4 group of ω chain), 2.02-2.06 (m, 2H, one proton of CH₂-4 group and one of CH₂-7 group of α chain), 2.11-2.15 (m, 3H, CH₂-2 of α chain and one proton of CH₂-4 group of α chain), 2.21 (m, 1H, one proton of CH₂-4 group of cyclopentyl ring), 2.29 (m, 1H, one proton of CH₂-7 group of α chain), 2.34 (m, 1H, CH-2cyclopentyl ring), 2.67 (m, 2H, CH₂-5 of ω chain), 3.22 (m, 2H, —CH₂CH₃), 3.55 (s, 3H, three —OH groups), 3.91 (m, 1H, CH-3 of cyclopentyl group), 4.08 (m, 1H, CH-3 of ω chain), 4.12 (m, 1H, CH-5 of cyclopentyl ring), 5.34 (m, 1H, CH-5 of α chain), 5.41 (m, 1H, CH-6 of α chain), 5.47 (dd, J=9.0 and 15.3 Hz, 1H, CH-1 of ω chain), 5.59 (dd, J=7.3 Hz and 15.3 Hz, 1H, CH-2 of ω chain), 5.98 (t, J=5.1 Hz, 1H, >NH), 7.17 (m, 1H, H-4 aromatic), 7.18 (m, 2H, H-2 and H-6 aromatic), 7.26 (m, 2H, H-3 and H-5 aromatic). ¹³C NMR (150 MHz, CDCl₃, 25° C.) δ (ppm): 14.77 (—CH₂CH₃), 25.38 (C-7 of α chain), 25.63 (C-3 of α chain), 26.70 (C-4 of α chain), 31.88 (C-5 of ω chain), 34.40 (—CH₂CH₃), 35.82 (C-2 of α chain), 38.75 (C-4 of ω chain), 42.93 (C-4 of cyclopentyl ring), 50.19 (C-1 of cyclopentyl ring), 55.47 (C-2 of cyclopentyl ring), 72.25 (C-3 of ω chain), 72.33 (C-5 of cyclopentyl ring), 77.67 (C-3 of cyclopentyl ring), 125.77 (C-4 aromatic), 128.35 (2C, C-3 and C-5 aromatic), 128.35 (2C, C-2 and C-6 aromatic), 142.0 (C-1 aromatic), 129.18 (C-6 of α chain), 129.66 (C-5 of α chain), 133.20 (C-1 of ω chain), 135.12 (C-2 of ω chain), 173.42 (C═O).

HRMS (ESI): HRMS (ESI): calculated for C₂₅H₃₇NO₄Na [M+Na]⁺ 438.26148. found 438.2632

HPLC: Kinetex XB-C18, 2.6 m, 150×4.6 mm, H₂O/CH₃CN (8:2, phase A)/H₂O/CH₃CN (1:1, phase B) in concentration gradient 90%-70%, 1.0 ml/min, Rₜ=21.44 min. (0.12%—(15R)-(+)-7b), Rₜ=21.85 min. (8.38%—(5E,15S)-isomer), Rₜ=22.56 min. (91.50%—(15S)-(+)-7a).

HPLC-MS (ESI): Kinetex XB-C18, 2.7 μm, kolumna 150×4.6 mm, (600 μl NH₃.H₂O:500 μl CH₃COOH:1 dm³ H₂O):CH₃CN (8:2, phase A)/(600 μl NH₃.H₂O:500 μl CH₃COOH:1 dm³ H₂O):CH₃CN (8:1, phase B) in concentration gradient 100%-75%, 1.0 ml/min, Rₜ=21.79 min. (m/z=416.3 [M+H]⁺ for (15R)-(+)-7b), Rₜ=22.33 min. (m/z=416.3 [M+H]⁺ for (5E,15S)-(+)-7a), Rₜ=22.91 min. (m/z=416.3 [M+H]⁺ dla (15S)-(+)-10a).

### (5Z)—N-ethyl-7-[(1R,2R,3R,5S)-3,5-Dihydroxy-2-[(1E,3R)-3-hydroxy-5-phenyl-1-pentenyl]-cyclopentyl]hept-5-enamid (7b)

Following the same procedure, using (3R)-(+)-11b ester (11.70 g, 23.85 mmol) 8.30 g of (3R)-(+)-7b amide was obtained (83.8% yield, 7a:7b:(5E,15R)-isomer=0.10%:91.84%:8.06%). [α]_{D}²⁰=+19.76° (c 1.0, CH₂Cl₂). FT-IR (thin film) νₘₐₓ (cm⁻¹): 3310, 3087, 3025, 2972, 2933, 1650, 1555, 1496, 1454, 1366, 1334, 1265, 1201, 1074, 1030, 970, 920, 849, 748, 700. ¹H NMR (CDCl₃, 600 MHz, 25° C.): 1.09 (t, J=7.2 Hz, 3H, —CH₂CH₃), 1.45 (m, 1H, CH-1 of cyclopentyl ring), 1.65 (m, 2H, CH₂-3 of α chain), 1.77 (m, 1H, one proton of CH₂-4 group of cyclopentyl ring), 1.84 (m, 2H, CH₂-4 of ω chain), 2.04-2.14 (m, 6H, CH₂-4 and one proton of CH₂-4 group of cyclopentyl ring, CH₂-2 and one proton of CH₂-7 group of α chain), 2.26 (m, 1H, one proton of CH₂-7 group of α chain), 2.33 (m, 1H, CH-2 cyclopentyl ring), 2.67 (m, 1H, one proton of CH₂-5 group of ω chain), 2.73 (m, 1H, one proton of CH₂-5 group of ω chain), 3.22 (m, 2H, —CH₂CH₃), 3.40 (br. s, 3H, three —OH groups), 3.93 (m, 1H, CH-3 of cyclopentyl ring), 4.10 (m, 1H, CH-3 of ω chain), 4.13 (m, 1H, CH-5 cyclopentyl ring), 5.35 (m, 1H, CH-5 of α chain), 5.42 (m, 1H, CH-6 of α chain), 5.52 (dd, J=8.7 and 15.3 Hz, 1H, CH-1 of ω chain), 5.61 (dd, J=6.3 Hz i 15.3 Hz, 1H, CH-2 of ω chain), 6.07 (t, J=5.1 Hz, 1H, >NH), 7.17 (m, 1H, H-4 aromatic), 7.19 (m, 2H, H-2 and H-6 aromatic), 7.26 (m, 2H, H-3 and H-5 aromatic). ¹³C NMR (150 MHz, CDCl₃, 25° C.) δ (ppm): 14.70 (—CH₂CH₃), 25.36 (C-7 of α chain), 25.54 (C-3 of α chain), 26.58 (C-4 of α chain), 31.78 (C-5 of ω chain), 34.34 (—CH₂CH₃), 35.71 (C-2 of α chain), 38.76 (C-4 of ω chain), 43.0 (C-4 cyclopentyl ring), 50.79 (C-1 cyclopentyl ring), 55.49 (C-2 cyclopentyl ring), 71.51 (C-3 of ω chain), 72.62 (C-5 cyclopentyl ring), 77.94 (C-3 cyclopentyl ring), 125.68 (C-4 aromatic), 128.27 (2C, C-3 and C-5 aromatic), 128.39 (2C, C-2 and C-6 aromatic), 142.03 (C-1 aromatic), 129.24 (C-6 of α chain), 129.50 (C-5 of a chain), 132.10 (C-1 of ω chain), 134.64 (C-2 of ω chain), 173.39 (C═O). HRMS (ESI): calculated for C₂H₃₇NO₄Na [M+Na]⁺ 438.26148. found 438.2620.

HPLC: Kinetex XB-C18, 2.6 μm, 150×4.6 mm, H₂O/CH₃CN (8:2, phase A)/H₂O/CH₃CN (1:1, phase B) in concentration gradient 90%-70%, 1.0 ml/min, Rₜ=20.68 min. (8.06%—(5E,15R)-isomer), Rₜ=21.44 min. (91.84%—(15R)-(+)-7b), Rₜ=22.56 min. (0.10%—(15S)-(+)-7a).

HPLC-MS (ESI): Kinetex XB-C18, 2.7 μm, column 150×4.6 mm, (600 μl NH₃.H₂O: 500 μl CH₃COOH:1 dm³ H₂O):CH₃CN (8:2, phase A)/(600 μl NH₃.H₂O:500 μl CH₃COOH:1 dm³ H₂O):CH₃CN (8:1, phase B) in concentration gradient 100%-75%, 1.0 ml/min, Rₜ=21.19 min. (m/z=416.3 [M+H]⁺ for (5E,15R)-isomer), Rₜ=21.79 min. (m/z=416.3 [M+H]⁺ for (15R)-(+)-7b), Rₜ=22.91 min. (m/z=416.3 [M+H]⁺ dla (15S)-(+)-7a).

1. A process for preparation of prostaglandin F_{2α} analogues bearing a 13,14-en-15-ol ω-chain having on a 15R or 15S optical configuration, represented by the general formula (I), 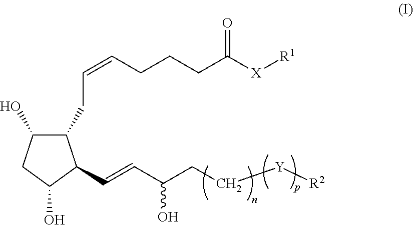
wherein:
X represents —O— or —NH—;
R¹ is H or C₁₋₃-alkyl;
Y represents —O—;
R² is H or phenyl group unsubstituted or substituted by trifluoromethyl group;
n represents an integer 0 or 1;
p represents an integer 0 or 1,
the process comprising the steps of:
(a) treatment of a phenylsulfone of the formula (II): 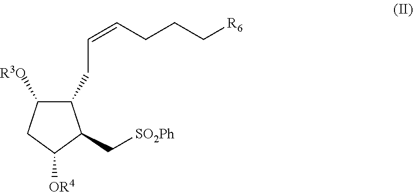
wherein:
R³ and R⁴ independently represent hydroxyl protecting group —Si(R⁹)(R¹⁰)(R¹¹), where and R⁹-R¹¹ are the same or different and are C₁₋₆-alkyl or phenyl;
R⁶ is an orthoester group, represented by the general formula (III), 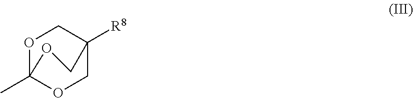
wherein R⁸ is H or C₁-C₆-alkyl,
or
R⁶ represents —C(OR¹²)₃ orthoester group, wherein R¹² is C₁-C₆-alkyl;
with a strong organometallic base to yield an α-sulfonyl carbanion of the phenylsulfone of the formula (II),
(b) addition of the α-sulfonyl carbanion in situ to an aldehyde having an optical configuration at a stereogenic center corresponding to 15R or 15S optical configuration of the target prostaglandin, respectively, the aldehyde being represented by the formula (IV), 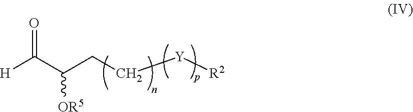
wherein:
R⁵ represents hydroxyl protecting group —Si(R⁹)(R¹⁰)(R¹¹), wherein R⁹-R¹¹ are the same or different and represent C₁₋₆-alkyl or phenyl; and
Y, R², n and p have the same meaning as defined for the formula (I),
to yield a mixture of diastereoisomers of β-hydroxysulfones of the general formula (V): 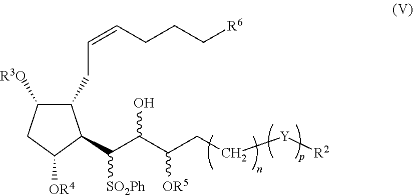
wherein R²-R⁶, Y, n and p have the same meaning as defined for the formula (I),
(c) reductive desulfonation of the mixture of β-hydroxysulfones of the general formula (V), to yield a compound having the 15R or 15S optical configuration and being represented by the formula (VI): 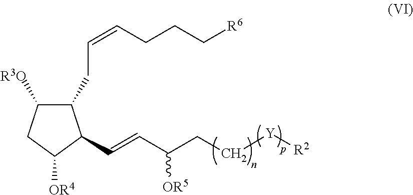
wherein R²-R⁶, Y, n and p have the same meaning as defined for the formula (I),
(d) removing R³, R⁴, R⁵ hydroxyl protecting groups to yield a compound having the 15R or 15S optical configuration and being represented by the formula (VII): 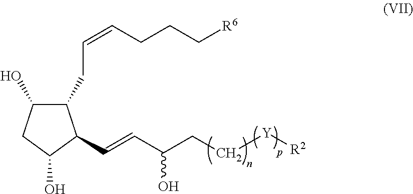
wherein
R², R⁶, Y, n and p have the same meaning as defined for the formula (I),
(e) hydrolysis of the compound of formula (VII) under acidic conditions, to yield a compound having the 15R or 15S optical configuration and being represented by the formula (VIII): 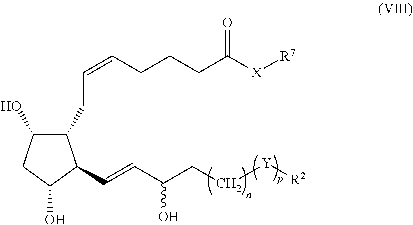
wherein:
X represents —O—;
R⁷ represents —CH₂—C(CH₂OH)₂—R⁸ or R¹² respectively; wherein R⁸ is H or C₁-C₆-alkyl and R¹² is C₁-C₆-alkyl; and
R², Y, n and p have the same meaning as defined for the formula (I),
(f) hydrolysis of the compound of formula (VIII) under basic conditions, to yield a compound having the 15R or 15S optical configuration and being represented by the formula (IA): 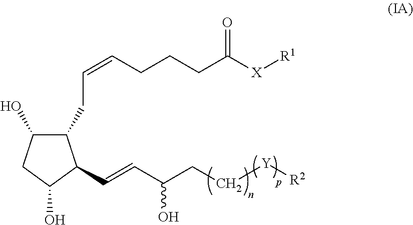
wherein:
X represents —O—;
R¹ is H; and
R², Y, n and p have the same meaning as defined for the formula (I),
and then
alkylating the compound of formula (IA) with C₁₋₃-alkyl halogen in the presence of a strong base, to obtain a compound having the 15R or 15S optical configuration and being represented by the formula (IB): 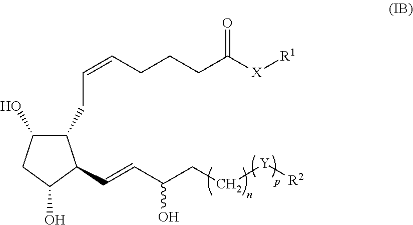
wherein:
X represents —O—;
R¹ is C₁₋₃-alkyl; and
R², Y, n and p have the same meaning as defined for the formula (I),
and, optionally,
reacting the compound of formula (IB) with an amine of the formula (IX):
R¹NH₂ (IX)
wherein R¹ is C₁₋₃-alkyl,
to obtain a prostamid having the 15R or 15S optical configuration and being represented by the formula (IC): 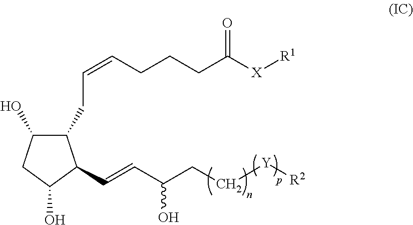
wherein:
X represents —NH—,
R¹ is C₁₋₃-alkyl; and
R², Y, n and p have the same meaning as defined for the formula (I);
or, optionally,
reacting the compound of formula (VIII) with the amine of the formula (IX):
R¹NH₂ (IX)
wherein R¹ is C₁₋₃-alkyl,
to obtain a prostamid having the 15R or 15S optical configuration and being represented by the formula (IC); 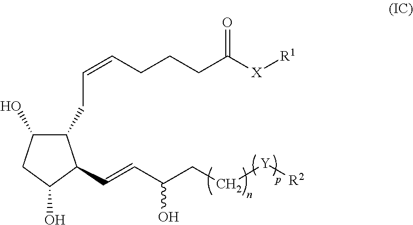
wherein:
X represents —NH—,
R¹ is C₁₋₃-alkyl; and
R², Y, n and p have the same meaning as defined for the formula (I).

2. The process of claim 1, wherein the α-sulfonyl carbanion of the formula (II) is generated by an alkali metal amide, selected from the group comprising lithium N,N-bis(trimethylsilyl)amide, sodium N,N-bis(trimethylsilyl)amide, lithium diisopropylamide and sodium diisopropylamide.

3. The process of claim 2, wherein the α-sulfonyl carbanion of the formula (II) is generated by lithium diisopropylamide.

4. The process of claim 1, wherein the desulfonation of the β-hydroxysulfones of the formula (V) is performed by using sodium amalgam in the presence of Na₂HPO₄ buffer.

5. The process of claim 1, wherein the R³-R⁵ groups are removed by reacting the compound of the formula (VI) with hydrogen fluoride or tetra-n-butylammonium fluoride.

6. The process of claim 1, wherein the R⁶ group of the compound of the formula (VII) is hydrolyzed in the presence of an aqueous solution of citric acid.

7. The process of claim 1, wherein the R⁷ group of the compound of the formula (VIII) is hydrolyzed with an alkali metal hydroxide, preferably with lithium hydroxide.

8. The process of claim 1, wherein the compound of formula (VIII) is alkylated with a C₁₋₃-alkyl halogen in the presence of a base, preferably 1,8-diazabicyclo[5.4.0]undec-7-en (DBU).

9. The process of claim 1, wherein the prostamid of the formula (IC), which is isopropyl ester of 16-[3-(trifluoromethoxy)phenoxy]-17,18,19,20-tetranor-prostaglandin F_{2α} (travoprost), is obtained in a diastereoisomeric excess greater than 99% de.

10. The process of claim 1, wherein the compound of formula (IB), which is 17-phenyl-18,19,20-trinorprostaglandin F_{2α} ethylamide (bimatoprost), is obtained in a diastereoisomeric excess greater than 99% de.

11. An intermediate in the process for preparation of prostaglandin F_{2α} analogues of claim 1, which is a β-hydroxysulfone of 15R or 15S configuration, the intermediate being represented by the formula (V): 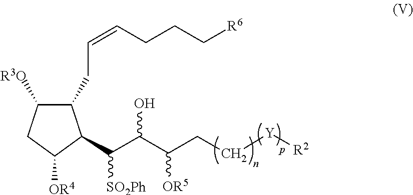
wherein:
R³, R⁴ and R⁵ independently represent —Si(R⁹)(R¹⁰)(R¹¹), wherein R⁹-R¹¹ are the same or different and represent C₁₋₆-alkyl or phenyl;
R⁶ is an orthoester, represented by the general formula (III), 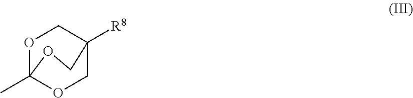
wherein R⁸ represents H or C₁-C₆-alkyl,
or
R⁶ represents —C(OR¹²)₃ orthoester group, wherein R¹² is C₁-C₆-alkyl;
Y represents —O—;
R² is H or phenyl unsubstituted or substituted by trifluoromethyl group;
n represents an integer 0 or 1; and
p represents an integer 0 or 1.

12. The intermediate of claim 11, wherein:
R³, R⁴ and R⁵ independently represent —Si(R⁹)(R¹⁰)(R¹¹), wherein R⁹-R¹¹ are the same or different and represent C₁₋₆-alkyl or phenyl;
R⁶ represents an orthoester, represented by the general formula (III), 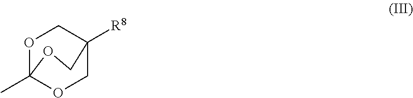
wherein R⁸ is H or C₁-C₆-alkyl,
and
when Y represents —O— and p=1, R² represents phenyl substituted in meta position by trifluoromethyl, and n=0;
and when Y represents —CH₂— and p=0, R² represents phenyl, and n=1.

13. An intermediate in the process for preparation of prostaglandin F_{2α} analogues of claim 1, the intermediate being represented by the formula (VI): 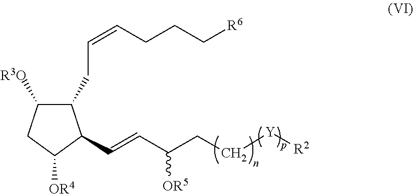
wherein:
R³, R⁴ and R⁵ independently represent —Si(R⁹)(R¹⁰)(R¹¹), wherein R⁹-R¹¹ are the same or different and represent C₁₋₆-alkyl or phenyl;
R⁶ represents an orthoester of the general formula (III), 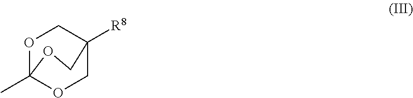
wherein R⁸ is H or C₁-C₆-alkyl,
or
R⁶ represents —C(OR¹²)₃ orthoester group, wherein R¹² is C₁-C₆-alkyl;
Y represents —O—;
R² is H or phenyl unsubstituted or substituted by trifluoromethyl;
n represents an integer, 0 or 1; and
p represents an integer, 0 or 1.

14. The intermediate of claim 12, wherein in the formula (VI):
R³, R⁴ and R⁵ independently represent —Si(R₉)(R₁₀)(R₁₁), wherein R₉-R₁₁ are the same or different and represent C₁₋₆-alkyl or phenyl;
R⁶ is the an orthoester of the general formula (III), 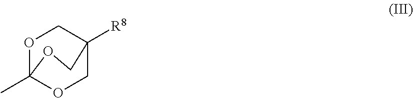
wherein
R⁸ represents H or C₁₋₆-alkyl;
and
when Y represents —O— and p=1, R² is phenyl substituted in meta position by trifluoromethyl, and n=0;
and when Y represents —CH₂— and p=0, R² is phenyl, and n=1.

15. An intermediate in the process for preparation of prostaglandin F_{2α} analogues of claim 1, the intermediate being represented by the formula (VII): 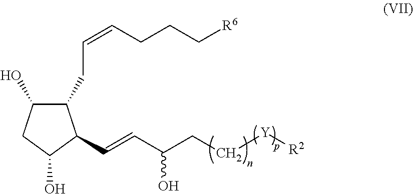
wherein:
R⁷ represents —CH₂—C(CH₂OH)₂—R group, wherein R⁸ is H or C₁-C₆-alkyl, or
R⁷ represents —C(OR¹²)₃ orthoester, wherein R¹² is C₁-C₆-alkyl;
Y represents —O—;
R² is H or phenyl unsubstituted or substituted by trifluoromethyl;
n represent an integer, 0 or 1; and
p represents an integer 0 or 1.

16. The intermediate of claim 15, wherein in the formula (VII):
R⁷ represents —CH₂—C(CH₂OH)₂—R group, wherein R⁸ is H or C₁-C₆-alkyl,
Y represents —O—;
and
when Y represents —O— and p=1, R² is phenyl substituted in meta position by trifluoromethyl, and n=0; and
when Y represents —CH₂— and p=0, R² is phenyl, and n=1.

17. An intermediate in the process for preparation of prostaglandin F_{2α} analogues of claim 1, the intermediate being represented by the formula (VIII): 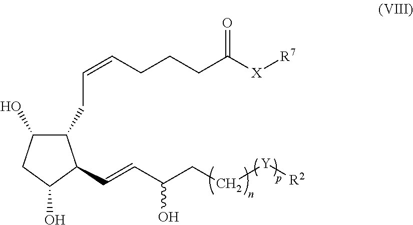
wherein:
X represents —O—;
R⁷ represents —CH₂—C(CH₂OH)₂—R⁸ or R¹² groups respectively, wherein R⁸ is H or C₁-C₆-alkyl, and R¹² is C₁-C₆-alkyl;
Y represents —O—;
R² is H or phenyl unsubstituted or substituted by trifluoromethyl;
n represents an integer, 0 or 1; and
p represents an integer, 0 or 1.

18. The intermediate of claim 17, represented by the formula (VIIIA) or (VIIIB): 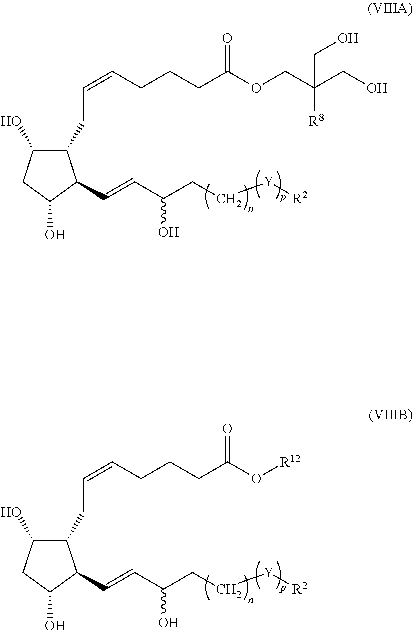
wherein:
R⁸ represents H or C₁₋₆-alkyl;
R¹² represents C₁₋₆-alkyl;
and
when Y represents —O— and p=1, R² is phenyl substituted in meta position by trifluoromethyl, and n=0;
and when Y represents —CH₂— and p=0, R² is phenyl, and n=1.

19. An aldehyde synthon for the process for preparation of prostaglandin F_{2α} analogues of claim 1, the aldehyde synthon having an optical configuration S or R at the stereogenic center and having an enantiomeric excess greater than 99% ee, the aldehyde synthon being represented by the formula (IV), 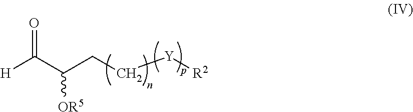
wherein:
Y represents —O—;
R² is H or phenyl group unsubstituted or substituted by trifluoromethyl group;
n represents an integer 0 or 1;
p represents an integer 0 or 1; and
R⁵ represents hydroxyl protecting group —Si(R⁹)(R¹⁰)(R¹¹), and R⁹-R¹¹ are the same or different and represent C₁₋₆-alkyl or phenyl.

20. The aldehyde synthon according to claim 19, wherein the aldehyde synthon is:
(S)-(−)-2-(tert-butyldimethylsililoxy)-3-(3-trifluoromethylphenoxy)propanal,
(R)-(+)-2-(tert-butyldimethylsililoxy)-3-(3-trifluoromethylphenoxy)propanal,
(S)-(−)-2-(tert-butyldimethylsililoxy)-4-phenylbutanal, or
(R)-(+)-2-(tert-butyldimethylosililoxy)-4-phenylbutanal.

21. A process for preparation of the aldehyde synthon of claim 19,
wherein
(a) a primary hydroxyl group of 1,2-diol of configuration at stereogenic center 2S or 2R having an enantiomeric excess greater than 99% ee, the 1,2-diol being represented by the formula (IV-1): 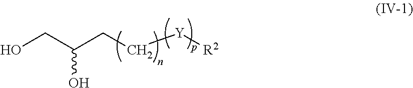
wherein R², Y, n and p have the meaning as defined for formula (IV), is selectively esterified with pivaloyl chloride under basic conditions, to obtain an α-hydroxypivaloate of formula (IV-2): 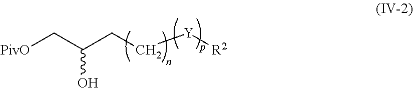
wherein R², Y, n and p have the meaning as defined for formula (IV),
(b) a secondary hydroxyl group of α-hydroxypivaloate of formula (IV-2) is protected by silylation with silyl chloride of formula R⁵C1,
wherein
R⁵ represents —Si(R⁹)(R¹⁰)(R¹¹), where and R⁹-R¹¹ are the same or different and represent C₁₋₆-alkyl or phenyl,
to obtain a compound of formula (IV-3), 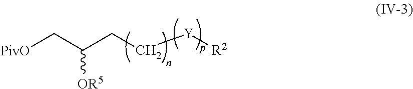
wherein:
R⁵ represents hydroxyl protecting group —Si(R⁹)(R¹⁰)(R¹¹), and R⁹-R¹¹ are the same or different and represent C₁₋₆-alkyl or phenyl, and
R², Y, n and p have the meaning as defined for formula (IV);
(c) the pivaloate ester of formula (IV-3) is deprotected with diisobutylaluminum hydride, to obtain an alcohol of formula (IV-4): 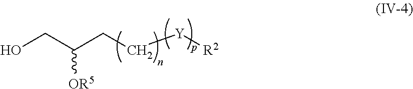
wherein:
R⁵ represents hydroxyl protecting group —Si(R⁹)(R¹⁰)(R¹¹), and R⁹-R¹¹ same or different and represent C₁₋₆-alkyl or phenyl, and
R², Y, n and p have the meaning as defined for formula (IV),
(d) the alcohol of formula (IV-4) is oxidized to obtain an aldehyde represented by formula (IV-5): 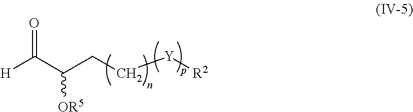
wherein:
R⁵ represents hydroxyl protecting group —Si(R⁹)(R¹⁰)(R¹¹), and R⁹-R¹¹ are the same or different and represent C₁₋₆-alkyl or phenyl, and
R², Y, n and p have the meaning as defined for formula (IV), and, optionally
(d) the protecting group R⁵ is removed to give an aldehyde of formula (IVA): 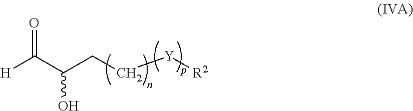
wherein R², Y, n and p have the meaning as defined for formula (IV).

22. The process according to claim 21, wherein the alcohol of formula (IV-4) is oxidized to the aldehyde of formula (IV-5) with a Dess-Martin reagent.
